(19) United States
(12) Patent Application Publication
(10) Pub. No.: US 2022/0168047 A1
(43) Pub. Date: Jun. 2, 2022

(54) MEDICAL ARM SYSTEM, CONTROL DEVICE, AND CONTROL METHOD

(71) Applicant: Sony Group Corporation, Tokyo (JP)

(72) Inventors: Daisuke NAGAO, Tokyo (JP); Yohei KURODA, Tokyo (JP)

(73) Assignee: Sony Group Corporation, Tokyo (JP)

(21) Appl. No.: 17/440,800

(22) PCT Filed: Mar. 19, 2020
(86) PCT No.: PCT/JP2020/012495

§ 371 Date: Sep. 20, 2021

(30) Foreign Application Priority Data
Mar. 27, 2019 (JP) 2019-059940

(51) Int. Cl. A61B 34/20; A61B 1/00; A61B 34/30; A61B 34/00

(52) U.S. Cl. CPC A61B 34/20; A61B 1/00149; A61B 34/30; A61B 2034/301; A61B 34/77; A61B 2034/2057; A61B 1/00006

(57) ABSTRACT

A medical arm system including: an arm unit that supports a medical instrument, and adapts a position and posture of the medical instrument with respect to a point of action on the medical instrument; and a control unit that controls an operation of the arm unit to adapt the position and the posture of the medical instrument with respect to the point of action and, one or more acquisition units configured to acquire environment information of a space surrounding the point of action, wherein the control unit is configured to generate or to update mapping information mapping the space surrounding the point of action on a basis of the environment information acquired by the one or more acquisition units and arm state information representing the position and the posture of the medical instrument with respect to the point of action according to a state of the arm unit.

### CROSS REFERENCE TO RELATED APPLICATIONS

This application claims the benefit of Japanese Priority Patent Application JP 2019-059940 filed on Mar. 27, 2019, the entire contents of which are incorporated herein by reference.

### TECHNICAL FIELD

The present disclosure relates to a medical arm system, a control device, and a control method.

### BACKGROUND ART

In recent years, in the medical field, methods of performing various operations such as surgery while observing an image of an operation site captured by an imaging device, using a balance-type arm (hereinafter referred to as “support arm”) having the imaging device held in a distal end of an arm, have been proposed. By using the balance-type arm, an affected part can be stably observed from a desired direction, and the operation can be efficiently performed. Examples of such an imaging device include an endoscope device and a microscope device.

Furthermore, in a case of observing an inside of a human body using an endoscope device, a situation where an obstacle exists in front of an observation target may occur. Under such circumstances, there are cases where the observation target can be observed without being blocked by the obstacle by using an oblique endoscope. As a specific example, PTL 1 discloses an example of a medical arm system assuming use of an oblique endoscope.

### CITATION LIST

### Patent Literature

PTL 1: WO 2018/159338

### SUMMARY

### Technical Problem

In the case of observing an inside of a human body using an endoscope device, it is desirable to control the position and posture of the endoscope device such that the observation target is located on an optical axis of an endoscope (lens barrel) attached to a camera head, for example. If a surgeon is only provided with an image captured by the endoscope device, it can be difficult to understand the situation around the endoscope device. As described above, under circumstances where it is difficult to understand the situation around a medical instrument such as the endoscope device or an arm supporting the medical instrument, a situation where a surgeon has a difficulty in operating the medical instrument as desired may occur.

Thus, the present disclosure proposes a technology for enabling control an operation of an arm in a more favorable form according to a surrounding situation.

### Solution to Problem

According to an embodiment of the present disclosure, provided is a medical arm system including: An arm unit configured to support a medical instrument, and to adapt a position and a posture of the medical instrument with respect to a point of action on the medical instrument; and a control unit configured to control an operation of the arm unit to adapt the position and the posture of the medical instrument with respect to the point of action and, one or more acquisition units configured to acquire environment information of a space surrounding the point of action, wherein the control unit is configured to generate or to update mapping information mapping the space surrounding the point of action on a basis of the environment information acquired by the one or more acquisition units and arm state information representing the position and the posture of the medical instrument with respect to the point of action according to a state of the arm unit.

It will be appreciated by a person skilled in the art that a point of action can be anywhere on a medical instrument. The point of action may correspond to a distal end of the medical instrument which enters a body cavity for example. Accordingly, the space surrounding the point of action may correspond to a surgical site for example.

Furthermore, according to an embodiment of the present disclosure, provided is a control device including: a control unit configured to control an operation of an arm unit to adapt a position and a posture of a medical instrument with respect to a point of action on the medical instrument, the arm unit being configured to support the medical instrument, and one or more acquisition units configured to acquire information of a space surrounding the point of action, wherein the control unit configured to generate or update mapping information mapping the space surrounding the point of action on a basis of environment information acquired by the one or more acquisition units and arm state information representing the position and the posture of the medical instrument with respect to the point of action according to a state of the arm unit.

Furthermore, according to an embodiment of the present disclosure, the control unit controls the operation of the arm unit on the basis of mapping information mapping a space surrounding the point of action.

Furthermore, according to an embodiment of the present disclosure, provided is a control method including: by a computer, controlling an arm unit to adapt a position and a posture of a medical instrument with respect to a point of action on the medical instrument, the arm unit being configured to support the medical instrument, acquiring environment information of a space surrounding the point of action, and generating or updating mapping information mapping the space surrounding the point of action on a basis of the environment information acquired by the acquisition unit and arm state information representing the position and the posture of the medical instrument with respect to the point of action according to a state of the arm unit.

Furthermore, according to an embodiment of the present disclosure, provided is a control method in which the operation of the arm unit is controlled on the basis of mapping information mapping a space around the point of action.

It will be appreciated that the phrase “adapt a position and a posture of the medical instrument” includes changing, controlling or altering the position and the posture of the medical instrument.

### BRIEF DESCRIPTION OF DRAWINGS

FIG. 1 is a diagram illustrating an example of a schematic configuration of an endoscopic surgical system to which the technology according to an embodiment of the present disclosure is applicable.

FIG. 2 is a block diagram illustrating an example of functional configurations of a camera head and a CCU illustrated in FIG. 1.

FIG. 3 is a schematic view illustrating an appearance of a support arm device according to the embodiment.

FIG. 4 is a schematic view illustrating a configuration of an oblique endoscope according to the embodiment.

FIG. 5 is a schematic view illustrating an oblique endoscope and a straight endoscope in comparison.

FIG. 6 is a functional block diagram illustrating a configuration example of a medical arm system according to the embodiment.

FIG. 7 is an explanatory diagram for describing an overview of an example of arm control in a case of performing an observation using an oblique endoscope.

FIG. 8 is an explanatory diagram for describing an overview of an example of arm control in a case of performing an observation using an oblique endoscope.

FIG. 9 is an explanatory diagram for describing an example of technical problems in a case of performing an observation using an oblique endoscope.

FIG. 10 is an explanatory diagram for describing an example of an effect obtained by using a polarization image sensor.

FIG. 11 is an explanatory diagram for describing an example of an effect obtained by using a polarization image sensor.

FIG. 12 is a flowchart illustrating an example of a flow of a series of processing of a control device according to the embodiment.

FIG. 13 is an explanatory diagram for describing an example of a schematic configuration of an endoscope device according to a first modification.

FIG. 14 is an explanatory diagram for describing an overview of an operation of a medical arm system according to a second modification.

FIG. 15 is an explanatory diagram for describing an overview of an operation of a medical arm system according to a third modification.

FIG. 16 is an explanatory diagram for describing an overview of an example of arm control according to a first example.

FIG. 17 is an explanatory diagram for describing an overview of another example of the arm control according to the first example.

FIG. 18 is an explanatory diagram for describing an overview of an example of arm control according to a second example.

FIG. 19 is an explanatory diagram for describing an overview of another example of the arm control according to the second example.

FIG. 20 is an explanatory diagram for describing an overview of an example of arm control according to a third example.

FIG. 21 is an explanatory diagram for describing an overview of another example of the arm control according to the third example.

FIG. 22 is an explanatory diagram for describing an overview of another example of arm control according to a fourth example.

FIG. 23 is an explanatory diagram for describing an overview of an example of arm control according to a fifth example.

FIG. 24 is an explanatory diagram for describing an example of control regarding generation or update of an environment map according to a seventh example.

FIG. 25 is an explanatory diagram for describing an example of control regarding generation or update of an environment map according to the seventh example.

FIG. 26 is an explanatory diagram for describing an example of control using a prediction model in a medical arm system according to an eighth example.

FIG. 27 is an explanatory diagram for describing an example of control using a prediction model in the medical arm system according to the eighth example.

FIG. 28 is a functional block diagram illustrating a configuration example of a hardware configuration of an information processing apparatus according to the embodiment.

FIG. 29 is an explanatory diagram for describing an application of a medical observation system according to the embodiment.

### DESCRIPTION OF EMBODIMENTS

Favorable embodiments of the present disclosure will be described in detail with reference to the appended drawings. Note that, in the present specification and drawings, redundant description of configuration elements having substantially the same functional configuration is omitted by providing the same sign.

Note that the description will be given in the following order.

1. Configuration Example of Endoscopic System

2. Configuration Example of Support Arm Device

3. Basic Configuration of Oblique Endoscope

4. Functional Configuration of Medical Arm System

5. Control of Arm

5.1. Overview

5.2. Environment Map Generation Method

5.3. Processing

5.4. Modification

5.5. Example

6. Hardware Configuration

7. Application

8. Conclusion

### 1. Configuration Example of Endoscopic System

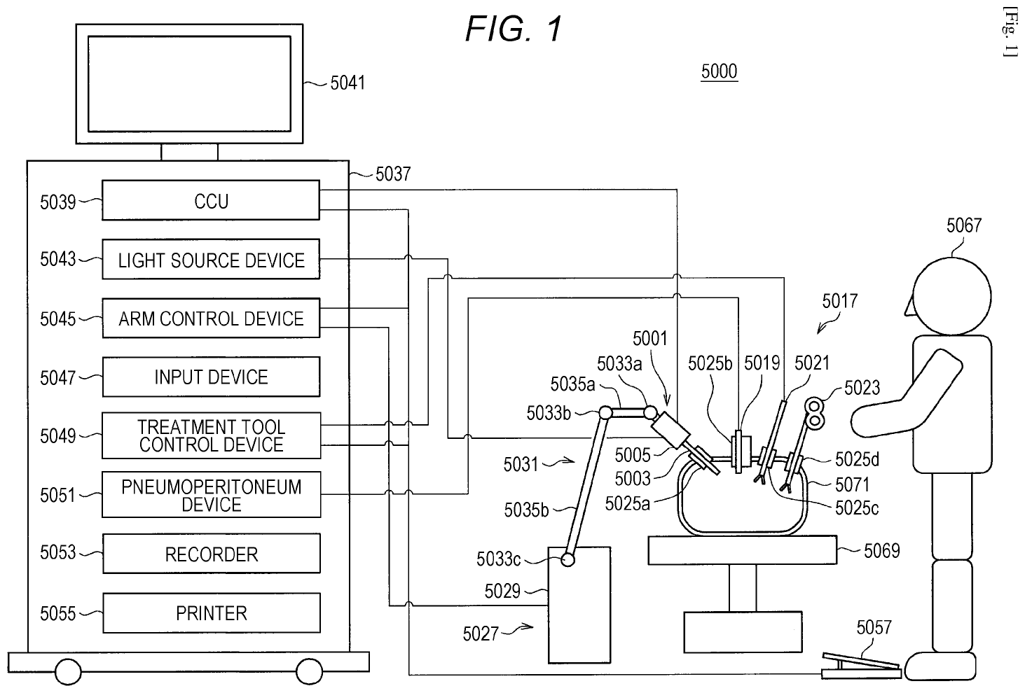

FIG. 1 is a diagram illustrating an example of a schematic configuration of an endoscopic surgical system 5000 to which the technology according to the present disclosure is applicable. FIG. 1 illustrates a state in which an operator (surgeon) 5067 is performing an operation on a patient 5071 on a patient bed 5069, using the endoscopic surgical system 5000. As illustrated, the endoscopic surgical system 5000 includes an endoscope device 5001, other surgical tools 5017, a support arm device 5027 that supports the endoscope device 5001, and a cart 5037 in which various devices for endoscopic surgery are mounted.

In laparoscopic surgery, a plurality of cylindrical puncture instruments called trocars 5025a to 5025d is punctured into an abdominal wall instead of cutting the abdominal wall and opening the abdomen. Then, a lens barrel 5003 (in other words, an endoscope unit) of the endoscope device 5001 and other surgical tools 5017 are inserted into a body cavity of the patient 5071 through the trocars 5025a to 5025d. In the illustrated example, as the other surgical tools 5017, a pneumoperitoneum tube 5019, an energy treatment tool 5021, and a forceps 5023 are inserted into the body cavity of the patient 5071. Furthermore, the energy treatment tool 5021 is a treatment tool for performing incision and detachment of tissue, sealing of a blood vessel, and the like with a high-frequency current or an ultrasonic vibration. Note that the illustrated surgical tools 5017 are mere examples, and various kinds of surgical tools typically used in endoscopic surgery such as tweezers and a retractor may be used as the surgical tool 5017.

An image of an operation site in the body cavity of the patient 5071 captured by the endoscope device 5001 is displayed on a display device 5041. The operator 5067 performs treatment such as removal of an affected part, for example, using the energy treatment tool 5021 and the forceps 5023 while viewing the image of the operation site displayed on the display device 5041 in real time. Note that the pneumoperitoneum tube 5019, the energy treatment tool 5021, and the forceps 5023 are supported by the operator 5067, an assistant, or the like during surgery, although illustration is omitted.

(Support Arm Device)

The support arm device 5027 includes an arm unit 5031 extending from a base unit 5029. In the illustrated example, the arm unit 5031 includes joint units 5033a, 5033b, and 5033c, and links 5035a and 5035b, and is driven under the control of an arm control device 5045. The endoscope device 5001 is supported by the arm unit 5031, and the position and posture of the endoscope device 5001 are controlled. With the control, stable fixation of the position of the endoscope device 5001 can be realized.

(Endoscope Device)

The endoscope device 5001 includes the lens barrel 5003 (endoscope unit) and a camera head 5005. A region having a predetermined length from a distal end of the lens barrel 5003 is inserted into the body cavity of the patient 5071. The camera head 5005 is connected to a proximal end of the lens barrel 5003. In the illustrated example, the endoscope device 5001 configured as a so-called hard endoscope including the hard lens barrel 5003 is illustrated. However, the endoscope device 5001 may be configured as a so-called soft endoscope including the soft lens barrel 5003.

An opening portion in which an object lens is fit is provided in the distal end of the lens barrel 5003 (endoscope unit). A light source device 5043 is connected to the endoscope device 5001, and light generated by the light source device 5043 is guided to the distal end of the lens barrel 5003 by a light guide extending inside the lens barrel 5003 and an observation target in the body cavity of the patient 5071 is irradiated with the light through the object lens. Note that the lens barrel 5003 connected to the camera head 5005 may a direct-viewing endoscope, an oblique endoscope, or a side endoscope.

An optical system and an imaging element are provided inside the camera head 5005, and reflected light (observation light) from the observation target is condensed to the imaging element by the optical system. The observation light is photoelectrically converted by the imaging element, and an electrical signal corresponding to the observation light, in other words, an image signal corresponding to an observed image is generated. The image signal is transmitted to a camera control unit (CCU) 5039 as raw data. Note that the camera head 5005 has a function to adjust magnification and a focal length by appropriately driving the optical system.

Note that a plurality of the imaging elements may be provided in the camera head 5005 to support three-dimensional (3D) display, and the like, for example. In this case, a plurality of relay optical systems is provided inside the lens barrel 5003 to guide the observation light to each of the plurality of imaging elements.

(Various Devices Mounted in Cart)

The CCU 5039 includes a central processing unit (CPU), a graphics processing unit (GPU), and the like, and centrally controls the operation of the endoscope device 5001 and the display device 5041. Specifically, the CCU 5039 receives the image signal from the camera head 5005, and applies various types of image processing for displaying an image based on the image signal, such as developing processing (demosaicing processing), for example, to the image signal. The CCU 5039 provides the image signal to which the image processing has been applied to the display device 5041. Furthermore, the CCU 5039 transmits a control signal to the camera head 5005 to control its driving. The control signal may include information regarding imaging conditions such as the magnification and focal length.

The display device 5041 displays an image based on the image signal to which the image processing has been applied by the CCU 5039, under the control of the CCU 5039. In a case where the endoscope device 5001 supports high-resolution capturing such as 4K (horizontal pixel number 3840×vertical pixel number 2160) or 8K (horizontal pixel number 7680×vertical pixel number 4320), and/or in a case where the endoscope device 5001 supports 3D display, for example, the display device 5041, which can perform high-resolution display and/or 3D display, can be used corresponding to each case. In a case where the endoscope device 5001 supports the high-resolution capturing such as 4K or 8K, a greater sense of immersion can be obtained by use of the display device 5041 with the size of 55 inches or more. Furthermore, a plurality of display devices 5041 having different resolutions and sizes may be provided depending on the application.

The light source device 5043 includes a light source such as a light emitting diode (LED) for example, and supplies irradiation light to the endoscope device 5001 in capturing an operation site.

The arm control device 5045 includes a processor such as a CPU, and is operated according to a predetermined program, thereby controlling drive of the arm unit 5031 of the support arm device 5027 according to a predetermined control method.

An input device 5047 is an input interface for the endoscopic surgical system 5000. The user can input various types of information and instructions to the endoscopic surgical system 5000 through the input device 5047. For example, the user inputs various types of information regarding surgery, such as patient's physical information and information of an operative procedure of the surgery, through the input device 5047. Furthermore, for example, the user inputs an instruction to drive the arm unit 5031, an instruction to change the imaging conditions (such as the type of the irradiation light, the magnification, and the focal length) of the endoscope device 5001, an instruction to drive the energy treatment tool 5021, or the like through the input device 5047.

The type of the input device 5047 is not limited, and the input device 5047 may be one of various known input devices. For example, a mouse, a keyboard, a touch panel, a switch, a foot switch 5057, a lever, and/or the like can be applied to the input device 5047. In a case where a touch panel is used as the input device 5047, the touch panel may be provided on a display surface of the display device 5041.

Alternatively, the input device 5047 is a device worn by the user, such as a glass-type wearable device or a head mounted display (HMD), for example, and various inputs are performed according to a gesture or a line of sight of the user detected by the device. Furthermore, the input device 5047 includes a camera capable of detecting a movement of the user, and various inputs are performed according to a gesture or a line of sight of the user detected from a video captured by the camera. Moreover, the input device 5047 includes a microphone capable of collecting a voice of the user, and various inputs are performed by an audio through the microphone. In this way, the input device 5047 is configured to be able to input various types of information in a non-contact manner, whereby the user (for example, the operator 5067) in particular belonging to a clean area can operate a device belonging to a filthy area in a non-contact manner. Furthermore, since the user can operate the device without releasing his/her hand from the possessed surgical tool, the user's convenience is improved.

A treatment tool control device 5049 controls drive of the energy treatment tool 5021 for cauterization and incision of tissue, sealing of a blood vessel, and the like. A pneumoperitoneum device 5051 sends a gas into the body cavity of the patient 5071 through the pneumoperitoneum tube 5019 to expand the body cavity for the purpose of securing a field of view by the endoscope device 5001 and a work space for the operator. A recorder 5053 is a device that can record various types of information regarding the surgery. A printer 5055 is a device that can print the various types of information regarding the surgery in various formats such as a text, an image, or a graph.

Hereinafter, a particularly characteristic configuration in the endoscopic surgical system 5000 will be further described in detail.

(Support Arm Device)

The support arm device 5027 includes the base unit 5029 as a base and the arm unit 5031 extending from the base unit 5029. In the illustrated example, the arm unit 5031 includes the plurality of joint units 5033a, 5033b, and 5033c and the plurality of links 5035a and 5035b connected by the joint unit 5033b, but FIG. 1 illustrates the configuration of the arm unit 5031 in a simplified manner to aid explanation. In reality, the shapes, the number, and the arrangement of the joint units 5033a to 5033c and the links 5035a and 5035b, the directions of rotation axes of the joint units 5033a to 5033c, and the like can be appropriately set so that the arm unit 5031 has a desired degree of freedom. For example, the arm unit 5031 can be favorably configured to have six degrees of freedom or more. With the configuration, the endoscope device 5001 can be freely moved within a movable range of the arm unit 5031. Therefore, the lens barrel 5003 of the endoscope device 5001 can be inserted from a desired direction into the body cavity of the patient 5071.

Actuators are provided in the joint units 5033a to 5033c, and the joint units 5033a to 5033c are configured to be rotatable around a predetermined rotation axis by driving of the actuators. The drive of the actuators is controlled by the arm control device 5045, whereby rotation angles of the joint units 5033a to 5033c are controlled and drive of the arm unit 5031 is controlled. With the control, control of the position and posture of the endoscope device 5001 can be realized. At this time, the arm control device 5045 can control the drive of the arm unit 5031 by various known control methods such as force control or position control.

For example, the drive of the arm unit 5031 may be appropriately controlled by the arm control device 5045 according to an operation input, and the position and posture of the endoscope device 5001 may be controlled, by an appropriate operation input by the operator 5067 via the input device 5047 (including the foot switch 5057). With the control, the endoscope device 5001 at the distal end of the arm unit 5031 can be moved from an arbitrary position to an arbitrary position, and then can be fixedly supported at the position after the movement. Note that the arm unit 5031 may be operated by a so-called master-slave system. In this case, the arm unit 5031 (slave device) can be remotely operated by the user via the input device 5047 (master device) installed at a position in the operating room separated from the slave device or a position separated from the operating room.

Furthermore, in a case where the force control is applied, the arm control device 5045 may perform so-called power assist control in which the arm control device 5045 receives an external force from the user and drives the actuators of the joint units 5033a to 5033c so that the arm unit 5031 is smoothly moved according to the external force. With the control, the user can move the arm unit 5031 with a relatively light force when moving the arm unit 5031 while being in direct contact with the arm unit 5031. Accordingly, the user can more intuitively move the endoscope device 5001 with a simpler operation, and the user's convenience can be improved.

Here, in endoscopic surgery, the endoscope device 5001 has been generally supported by a surgeon called scopist. In contrast, by use of the support arm device 5027, the position of the endoscope device 5001 can be reliably fixed without manual operation, and thus an image of the operation site can be stably obtained and the surgery can be smoothly performed.

Note that the arm control device 5045 is not necessarily provided in the cart 5037. Furthermore, the arm control device 5045 is not necessarily one device. For example, the arm control device 5045 may be provided in each of the joint units 5033a to 5033c of the arm unit 5031 of the support arm device 5027, and the drive control of the arm unit 5031 may be realized by mutual cooperation of the plurality of arm control devices 5045.

(Light Source Device)

The light source device 5043 supplies irradiation light, which is used in capturing an operation site, to the endoscope device 5001. The light source device 5043 includes, for example, an LED, a laser light source, or a white light source configured by a combination thereof. In a case where the white light source is configured by a combination of RGB laser light sources, output intensity and output timing of the respective colors (wavelengths) can be controlled with high accuracy. Therefore, white balance of a captured image can be adjusted in the light source device 5043. Further, in this case, the observation target is irradiated with the laser light from each of the RGB laser light sources in a time division manner, and the drive of the imaging element of the camera head 5005 is controlled in synchronization with the irradiation timing, so that images respectively corresponding to RGB can be captured in a time division manner. According to the method, a color image can be obtained without providing a color filter to the imaging element.

Furthermore, drive of the light source device 5043 may be controlled to change intensity of light to be output every predetermined time. The drive of the imaging element of the camera head 5005 is controlled in synchronization with change timing of the intensity of light, and images are acquired in a time division manner and are synthesized, whereby a high-dynamic range image without blocked up shadows and flared highlights can be generated.

Further, the light source device 5043 may be configured to be able to supply light in a predetermined wavelength band corresponding to special light observation. In the special light observation, for example, so-called narrow band imaging is performed by radiating light in a narrower band than the irradiation light (in other words, white light) at the time of normal observation, using wavelength dependence of absorption of light in a body tissue, to capture a predetermined tissue such as a blood vessel in a mucosal surface layer at high contrast. Alternatively, in the special light observation, fluorescence observation to obtain an image by fluorescence generated by radiation of exciting light may be performed. In the fluorescence observation, irradiating the body tissue with exciting light to observe fluorescence from the body tissue (self-fluorescence observation), injecting a reagent such as indocyanine green (ICG) into the body tissue and irradiating the body tissue with exciting light corresponding to a fluorescence wavelength of the reagent to obtain a fluorescence image, or the like can be performed. The light source device 5043 can be configured to be able to supply narrow-band light and/or exciting light corresponding to such special light observation.

(Camera Head and CCU)

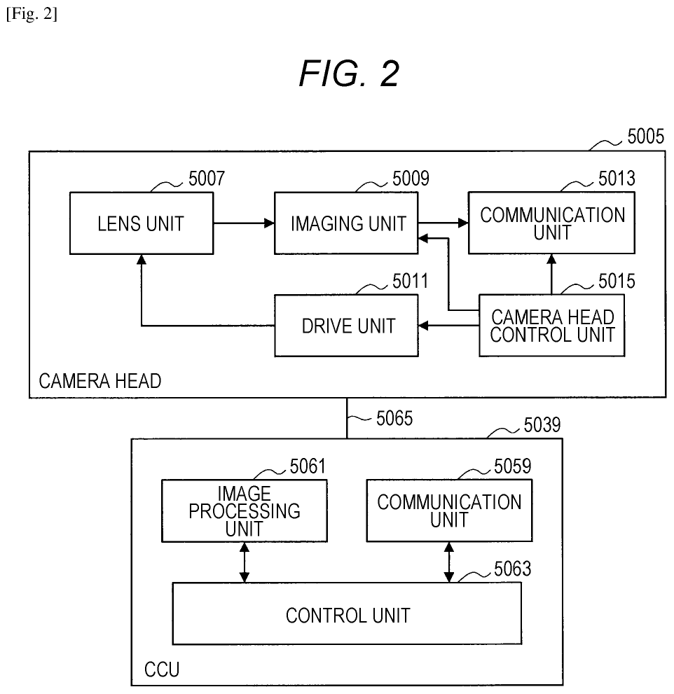

Functions of the camera head 5005 and the CCU 5039 of the endoscope device 5001 will be described in more detail with reference to FIG. 2. FIG. 2 is a block diagram illustrating an example of functional configurations of the camera head 5005 and the CCU 5039 illustrated in FIG. 1.

Referring to FIG. 2, the camera head 5005 includes a lens unit 5007, an imaging unit 5009, a drive unit 5011, a communication unit 5013, and a camera head control unit 5015 as its functions. Furthermore, the CCU 5039 includes a communication unit 5059, an image processing unit 5061, and a control unit 5063 as its functions. The camera head 5005 and the CCU 5039 are communicatively connected with each other by a transmission cable 5065.

First, a functional configuration of the camera head 5005 will be described. The lens unit 5007 is an optical system provided in a connection portion between the lens unit 5007 and the lens barrel 5003. Observation light taken through the distal end of the lens barrel 5003 is guided to the camera head 5005 and enters the lens unit 5007. The lens unit 5007 is configured by a combination of a plurality of lenses including a zoom lens and a focus lens. Optical characteristics of the lens unit 5007 are adjusted to condense the observation light on a light receiving surface of an imaging element of the imaging unit 5009. Furthermore, the zoom lens and the focus lens are configured to have their positions on the optical axis movable for adjustment of the magnification and focal point of the captured image.

The imaging unit 5009 includes an imaging element, and is disposed at a rear stage of the lens unit 5007. The observation light having passed through the lens unit 5007 is focused on the light receiving surface of the imaging element, and an image signal corresponding to the observed image is generated by photoelectric conversion. The image signal generated by the imaging unit 5009 is provided to the communication unit 5013.

As the imaging element constituting the imaging unit 5009, for example, a complementary metal oxide semiconductor (CMOS)-type image sensor having a Bayer array capable of color capturing is used. Note that, as the imaging element, for example, an imaging element that can capture a high-resolution image of 4K or more may be used. By obtainment of the image of the operation site with high resolution, the operator 5067 can grasp the state of the operation site in more detail and can more smoothly advance the surgery.

Furthermore, the imaging element constituting the imaging unit 5009 includes a pair of imaging elements for respectively obtaining image signals for right eye and for left eye corresponding to 3D display. With the 3D display, the operator 5067 can more accurately grasp the depth of biological tissue in the operation site. Note that, in a case where the imaging unit 5009 is configured as a multi-plate imaging unit, a plurality of systems of the lens units 5007 is provided corresponding to the imaging elements.

Furthermore, the imaging unit 5009 may not be necessarily provided in the camera head 5005. For example, the imaging unit 5009 may be provided immediately after the object lens inside the lens barrel 5003.

The drive unit 5011 includes an actuator, and moves the zoom lens and the focus lens of the lens unit 5007 by a predetermined distance along an optical axis by the control of the camera head control unit 5015. With the movement, the magnification and focal point of the captured image by the imaging unit 5009 can be appropriately adjusted.

The communication unit 5013 includes a communication device for transmitting or receiving various types of information to or from the CCU 5039. The communication unit 5013 transmits the image signal obtained from the imaging unit 5009 to the CCU 5039 through the transmission cable 5065 as raw data. At this time, to display the captured image of the operation site with low latency, the image signal is favorably transmitted by optical communication. This is because, in surgery, the operator 5067 performs surgery while observing the state of the affected part with the captured image, and thus display of a moving image of the operation site in as real time as possible is demanded for more safe and reliable surgery. In the case of the optical communication, a photoelectric conversion module that converts an electrical signal into an optical signal is provided in the communication unit 5013. The image signal is converted into the optical signal by the photoelectric conversion module, and is then transmitted to the CCU 5039 via the transmission cable 5065.

Furthermore, the communication unit 5013 receives a control signal for controlling drive of the camera head 5005 from the CCU 5039. The control signal includes information regarding the imaging conditions such as information for specifying a frame rate of the captured image, information for specifying an exposure value at the time of imaging, and/or information for specifying the magnification and the focal point of the captured image, for example. The communication unit 5013 provides the received control signal to the camera head control unit 5015. Note that the control signal from that CCU 5039 may also be transmitted by the optical communication. In this case, the communication unit 5013 is provided with a photoelectric conversion module that converts an optical signal into an electrical signal, and the control signal is converted into an electrical signal by the photoelectric conversion module and is then provided to the camera head control unit 5015.

Note that the imaging conditions such as the frame rate, exposure value, magnification, and focal point are automatically set by the control unit 5063 of the CCU 5039 on the basis of the acquired image signal. That is, a so-called auto exposure (AE) function, an auto focus (AF) function, and an auto white balance (AWB) function are incorporated in the endoscope device 5001.

The camera head control unit 5015 controls the drive of the camera head 5005 on the basis of the control signal received from the CCU 5039 through the communication unit 5013. For example, the camera head control unit 5015 controls drive of the imaging element of the imaging unit 5009 on the basis of the information for specifying the frame rate of the captured image and/or the information for specifying exposure at the time of imaging. Furthermore, for example, the camera head control unit 5015 appropriately moves the zoom lens and the focus lens of the lens unit 5007 via the drive unit 5011 on the basis of the information for specifying the magnification and focal point of the captured image. The camera head control unit 5015 may further have a function to store information for identifying the lens barrel 5003 and the camera head 5005.

Note that the configuration of the lens unit 5007, the imaging unit 5009, and the like is arranged in a hermetically sealed structure having high airtightness and waterproofness, whereby the camera head 5005 can have resistance to autoclave sterilization processing.

Next, a functional configuration of the CCU 5039 will be described. The communication unit 5059 includes a communication device for transmitting or receiving various types of information to or from the camera head 5005. The communication unit 5059 receives the image signal transmitted from the camera head 5005 through the transmission cable 5065. At this time, as described above, the image signal can be favorably transmitted by the optical communication. In this case, the communication unit 5059 is provided with a photoelectric conversion module that converts an optical signal into an electrical signal, corresponding to the optical communication. The communication unit 5059 provides the image signal converted into the electrical signal to the image processing unit 5061.

Furthermore, the communication unit 5059 transmits a control signal for controlling drive of the camera head 5005 to the camera head 5005. The control signal may also be transmitted by the optical communication.

The image processing unit 5061 applies various types of image processing to the image signal as raw data transmitted from the camera head 5005. The image processing include various types of known signal processing such as development processing, high image quality processing (such as band enhancement processing, super resolution processing, noise reduction (NR) processing, and/or camera shake correction processing), and/or enlargement processing (electronic zoom processing), for example. Furthermore, the image processing unit 5061 performs wave detection processing for image signals for performing AE, AF, and AWB.

The image processing unit 5061 is configured by a processor such as a CPU or a GPU, and the processor is operated according to a predetermined program, whereby the above-described image processing and wave detection processing can be performed. Note that in a case where the image processing unit 5061 includes a plurality of GPUs, the image processing unit 5061 appropriately divides the information regarding the image signal and performs the image processing in parallel by the plurality of GPUs.

The control unit 5063 performs various types of control related to imaging of the operation site by the endoscope device 5001 and display of the captured image. For example, the control unit 5063 generates a control signal for controlling drive of the camera head 5005. At this time, in a case where the imaging conditions are input by the user, the control unit 5063 generates the control signal on the basis of the input by the user. Alternatively, in a case where the AE function, the AF function, and the AWB function are incorporated in the endoscope device 5001, the control unit 5063 appropriately calculates optimum exposure value, focal length, and white balance according to a result of the wave detection processing by the image processing unit 5061, and generates the control signal.

Furthermore, the control unit 5063 displays the image of the operation site on the display device 5041 on the basis of the image signal to which the image processing has been applied by the image processing unit 5061. At this time, the control unit 5063 recognizes various objects in the image of the operation site, using various image recognition technologies. For example, the control unit 5063 can recognize a surgical instrument such as forceps, a specific living body portion, blood, mist at the time of use of the energy treatment tool 5021, or the like, by detecting a shape of an edge, a color or the like of an object included in the operation site image. The control unit 5063 superimposes and displays various types of surgery support information on the image of the operation site, in displaying the image of the operation site on the display device 5041 using the result of recognition. The surgery support information is superimposed, displayed, and presented to the operator 5067, so that the surgery can be more safely and reliably advanced.

The transmission cable 5065 that connects the camera head 5005 and the CCU 5039 is an electrical signal cable supporting communication of electrical signals, an optical fiber supporting optical communication, or a composite cable thereof.

Here, in the illustrated example, the communication has been performed in a wired manner using the transmission cable 5065. However, the communication between the camera head 5005 and the CCU 5039 may be wirelessly performed. In a case where the communication between the camera head 5005 and the CCU 5039 is wirelessly performed, it is unnecessary to lay the transmission cable 5065 in the operating room. Therefore, the situation in which movement of medical staff in the operating room is hindered by the transmission cable 5065 can be eliminated.

The example of the endoscopic surgical system 5000 to which the technology according to the present disclosure is applicable has been described. Note that, here, the endoscopic surgical system 5000 has been described as an example. However, a system to which the technology according to the present disclosure is applicable is not limited to this example. For example, the technology according to the present disclosure may be applied to a flexible endoscopic system for examination or a microsurgical system.

### 2. Configuration Example of Support Arm Device

Next, an example of a configuration of the support arm device to which the technology according to the present disclosure can be applied will be described below. The support arm device described below is an example configured as a support arm device that supports an endoscope at a distal end of an arm unit. However, the present embodiment is not limited to the example. Furthermore, in a case where the support arm device according to the embodiment of the present disclosure is applied to the medical field, the support arm device can function as a medical support arm device.

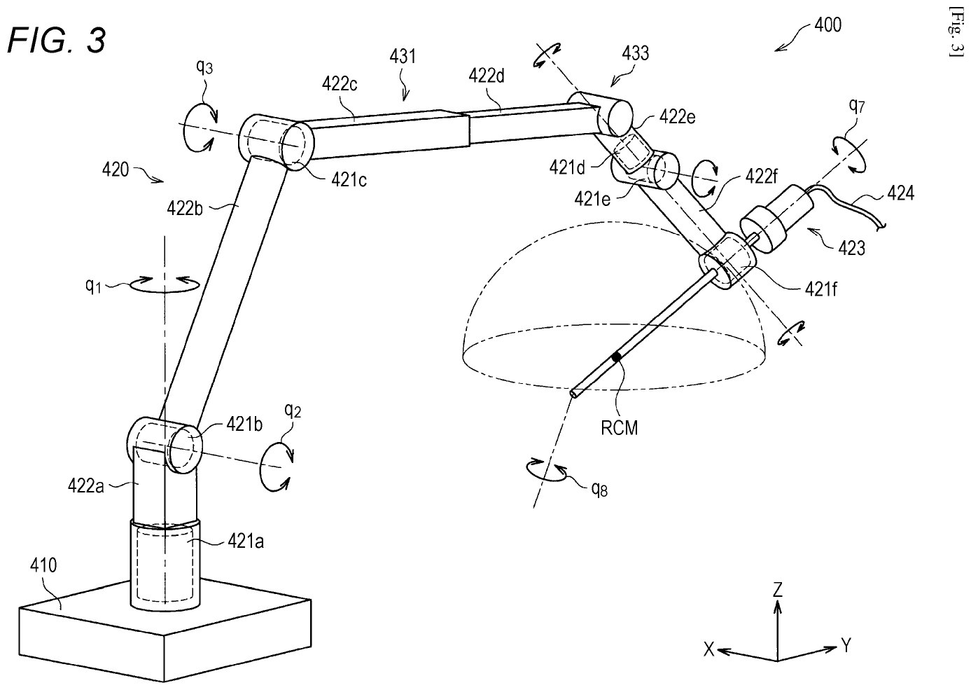

FIG. 3 is a schematic view illustrating an appearance of a support arm device 400 according to the present embodiment. As illustrated in FIG. 3, the support arm device 400 according to the present embodiment includes a base unit 410 and an arm unit 420. The base unit 410 is a base of the support arm device 400, and the arm unit 420 is extended from the base unit 410. Furthermore, although not illustrated in FIG. 3, a control unit that integrally controls the support arm device 400 may be provided in the base unit 410, and drive of the arm unit 420 may be controlled by the control unit. The control unit includes various signal processing circuits, such as a CPU and a DSP, for example.

The arm unit 420 includes a plurality of active joint units 421a to 421f, a plurality of links 422a to 422f, and an endoscope device 423 as a distal end unit provided at a distal end of the arm unit 420.

The links 422a to 422f are substantially rod-like members. One end of the link 422a is connected to the base unit 410 via the active joint unit 421a, the other end of the link 422a is connected to one end of the link 422b via the active joint unit 421b, and the other end of the link 422b is connected to one end of the link 422c via the active joint unit 421c. The other end of the link 422c is connected to the link 422d via a passive slide mechanism 431, and the other end of the link 422d is connected to one end of the link 422e via a passive joint unit 200. The other end of the link 422e is connected to one end of the link 422f via the active joint units 421d and 421e. The endoscope device 423 is connected to the distal end of the arm unit 420, in other words, the other end of the link 422f, via the active joint unit 421f. The respective ends of the plurality of links 422a to 422f are connected one another by the active joint units 421a to 421f, the passive slide mechanism 431, and the passive joint unit 433 with the base unit 410 as a fulcrum, as described above, so that an arm shape extended from the base unit 410 is configured.

Actuators provided in the respective active joint units 421a to 421f of the arm unit 420 are driven and controlled, so that the position and posture of the endoscope device 423 are controlled. In the present embodiment, the endoscope device 423 has a distal end enter a body cavity of a patient, which is an operation site, and captures a partial region of the operation site. However, the distal end unit provided at the distal end of the arm unit 420 is not limited to the endoscope device 423, and an external endoscope can be used instead of the endoscope. Furthermore, various medical instruments may be connected to the distal end of the arm unit 420 as the distal end unit. Thus, the support arm device 400 according to the present embodiment is configured as a medical support arm device provided with a medical instrument.

Here, hereinafter, the support arm device 400 will be described by defining coordinate axes as illustrated in FIG. 3. Furthermore, an up-down direction, a front-back direction, and a right-left direction will be defined in accordance with the coordinate axes. In other words, the up-down direction with respect to the base unit 410 installed on a floor is defined as a z-axis direction and the up-down direction Furthermore, a direction orthogonal to the z axis and in which the arm unit 420 is extended from the base unit 410 (in other words, a direction in which the endoscope device 423 is located with respect to the base unit 410) is defined as a y-axis direction and the front-back direction. Moreover, a direction orthogonal to the y axis and the z axis is defined as an x-axis direction and the right-left direction.

The active joint units 421a to 421f rotatably connect the links to one another. The active joint units 421a to 421f include actuators, and have a rotation mechanism that is rotationally driven about a predetermined rotation axis by drive of the actuators. By controlling rotational drive of each of the active joint units 421a to 421f, drive of the arm unit 420 such as extending or contracting (folding) of the arm unit 420 can be controlled, for example. Here, the drive of the active joint units 421a to 421f can be controlled by, for example, known whole body coordination control and ideal joint control. As described above, since the active joint units 421a to 421f have the rotation mechanism, in the following description, the drive control of the active joint units 421a to 421f specifically means control of rotation angles and/or generated torque (torque generated by the active joint units 421a to 4210 of the active joint units 421a to 421f.

The passive slide mechanism 431 is an aspect of a passive form change mechanism, and connects the link 422c and the link 422d to be able to move forward and backward along a predetermined direction. For example, the passive slide mechanism 431 may connect the link 422c and the link 422d in a linearly movable manner. However, the forward/backward motion of the link 422c and the link 422d is not limited to the linear motion, and may be forward/backward motion in a direction of forming an arc. The passive slide mechanism 431 is operated in the forward/backward motion by a user, for example, and makes a distance between the active joint unit 421c on the one end side of the link 422c and the passive joint unit 433 variable. Thereby, the entire form of the arm unit 420 can change.

The passive joint unit 433 is one aspect of the passive form change mechanism, and rotatably connects the link 422d and the link 422e to each other. The passive joint unit 433 is rotatably operated by the user, for example, and makes an angle made by the link 422d and the link 422e variable. Thereby, the entire form of the arm unit 420 can change.

Note that, in the present specification, the “posture of the arm unit” indicates a state of the arm unit in which at least a part of a portion configuring an arm is changeable by drive control or the like. As a specific example, a state of the arm unit changeable by the drive control of the actuators provided in the active joint units 421a to 421f by the control unit in a state where the distance between active joint units adjacent across one or a plurality of links is constant corresponds to the “posture of the arm unit”. Furthermore, a “form of the arm unit” indicates a state of the arm unit changeable as a relationship between the positions or postures of parts configuring an arm changes. As a specific example, a state of the arm unit changeable as the distance between active joint units adjacent across a link or an angle between links connecting adjacent active joint units changes with the operation of the passive form change mechanism corresponds to the “form of the arm unit”.

The support arm device 400 according to the present embodiment includes the six active joint units 421a to 421f and realizes six degrees of freedom with respect to the drive of the arm unit 420. That is, while the drive control of the support arm device 400 is realized by the drive control of the six active joint units 421a to 421f by the control unit, the passive slide mechanism 431 and the passive joint unit 433 are not the targets of the drive control by the control unit.

Specifically, as illustrated in FIG. 3, the active joint units 421a, 421d, and 421f are provided to have long axis directions of the connected links 422a and 422e and a capture direction of the connected endoscope device 423 as rotation axis directions. The active joint units 421b, 421c, and 421e are provided to have the x-axis direction that is a direction in which connection angles of the connected links 422a to 422c, 422e, and 422f and the connected endoscope device 423 are changed in a y-z plane (a plane defined by the y axis and the z axis) as rotation axis directions. As described above, in the present embodiment, the active joint units 421a, 421d, and 421f have a function to perform so-called yawing, and the active joint units 421b, 421c, and 421e have a function to perform so-called pitching.

With the above configuration of the arm unit 420, the support arm device 400 according to the present embodiment realizes the six degrees of freedom with respect to the drive of the arm unit 420, whereby freely moving the endoscope device 423 within the movable range of the arm unit 420. FIG. 3 illustrates a hemisphere as an example of a movable range of the endoscope device 423. In a case where a central point RCM (remote motion center) of the hemisphere is a capture center of the operation site captured by the endoscope device 423, the operation site can be captured from various angles by moving the endoscope device 423 on a spherical surface of the hemisphere in a state where the capture center of the endoscope device 423 is fixed to the central point of the hemisphere.

The example of the configuration of the support arm device to which the technology according to the present disclosure can be applied has been described.

### 3. Basic Configuration of Oblique Endoscope

Next, a basic configuration of an oblique endoscope will be described as an example of the endoscope.

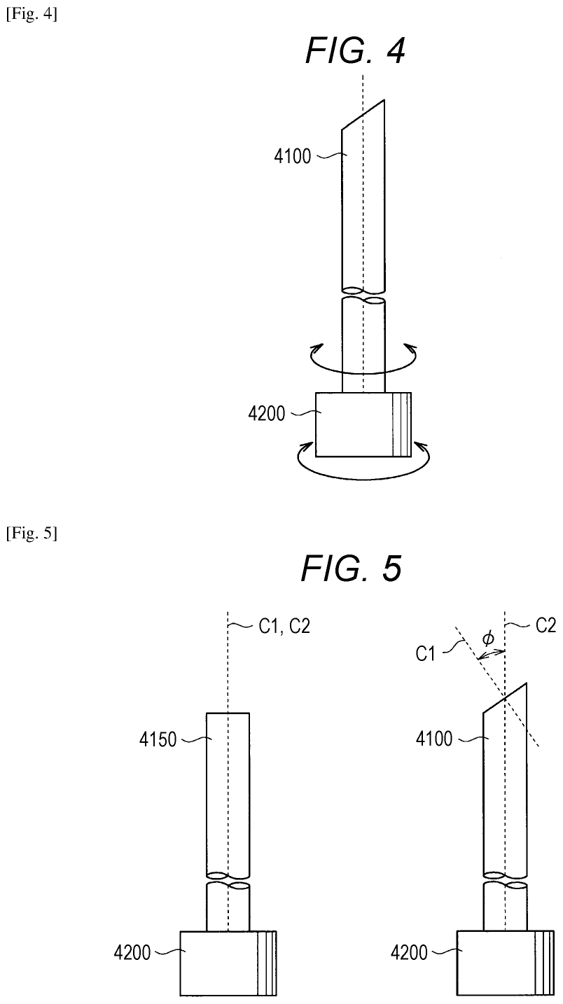

FIG. 4 is a schematic view illustrating a configuration of an oblique endoscope 4100 according to an embodiment of the present disclosure. As illustrated in FIG. 4, the oblique endoscope 4100 is attached to a distal end of a camera head 4200. The oblique endoscope 4100 corresponds to the lens barrel 5003 described in FIGS. 1 and 2, and the camera head 4200 corresponds to the camera head 5005 described in FIGS. 1 and 2. The oblique endoscope 4100 and the camera head 4200 may be rotatable independently of each other. An actuator may be provided between the oblique endoscope 4100 and the camera head 4200, similarly to the joint units 5033a, 5033b, and 5033c, and the oblique endoscope 4100 can rotate with respect to the camera head 4200 by drive of the actuator. Thereby, a rotation angle θ_{Z} described below is controlled.

The oblique endoscope 4100 is supported by a support arm device 5027. The support arm device 5027 has a function to hold the oblique endoscope 4100 instead of the scopist and to allow the oblique endoscope 4100 to be moved by an operation of the operator or the assistant so that a desired site can be observed.

FIG. 5 is a schematic view illustrating the oblique endoscope 4100 and a straight endoscope 4150 in comparison. In the straight endoscope 4150, a direction (C1) of an objective lens toward a subject coincides with a longitudinal direction (C2) of the straight endoscope 4150. On the other hand, in the oblique endoscope 4100, the direction (C1) of the objective lens toward the subject has a predetermined angle φ with respect to the longitudinal direction (C2) of the oblique endoscope 4100.

The basic configuration of the oblique endoscope has been described as an example of the endoscope.

### 4. Functional Configuration of Medical Arm System

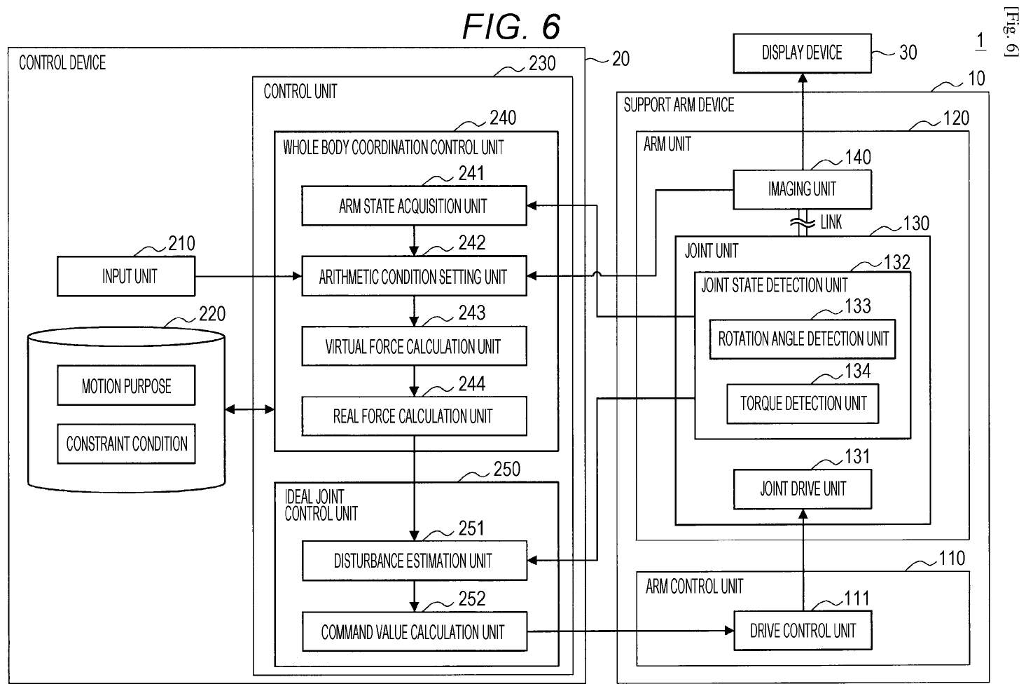

Next, a configuration example of a medical arm system according to an embodiment of the present disclosure will be described with reference to FIG. 6. FIG. 6 is a functional block diagram illustrating a configuration example of a medical arm system according to an embodiment of the present disclosure. Note that, in the medical arm system illustrated in FIG. 6, a configuration related to drive control of an arm unit of a support arm device will be mainly illustrated.

Referring to FIG. 6, a medical arm system 1 according to an embodiment of the present disclosure includes a support arm device 10, a control device 20, and a display device 30. In the present embodiment, the control device 20 performs various operations in accordance with the state of the arm unit of the support arm device 10, and controls the drive of the arm unit on the basis of operation results. Furthermore, the arm unit of the support arm device 10 holds an imaging unit 140, and an image captured by the imaging unit 140 is displayed on a display screen of the display device 30. Hereinafter, configurations of the support arm device 10, the control device 20, and the display device 30 will be described in detail.

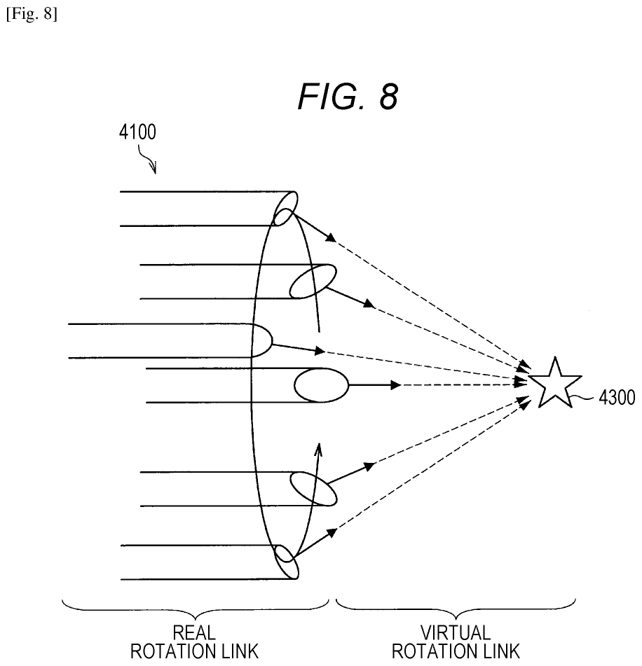

The support arm device 10 includes the arm unit that is a multilink structure including a plurality of joint units and a plurality of links, and drives the arm unit within a movable range to control the position and posture of the distal end unit provided at the distal end of the arm unit. The support arm device 10 corresponds to the support arm device 400 illustrated in FIG. 8.

Referring to FIG. 6, the support arm device 10 includes an arm control unit 110 and an arm unit 120. Furthermore, the arm unit 120 includes a joint unit 130 and the imaging unit 140.

The arm control unit 110 integrally controls the support arm device 10 and controls drive of the arm unit 120. Specifically, the arm control unit 110 includes a drive control unit 111. Drive of the joint unit 130 is controlled by the control of the drive control unit 111, so that the drive of the arm unit 120 is controlled. More specifically, the drive control unit 111 controls a current amount to be supplied to a motor in an actuator of the joint unit 130 to control the number of rotations of the motor, thereby controlling a rotation angle and generated torque in the joint unit 130. Note that, as described above, the drive control of the arm unit 120 by the drive control unit 111 is performed on the basis of the operation result in the control device 20. Therefore, the current amount to be supplied to the motor in the actuator of the joint unit 130, which is controlled by the drive control unit 111, is a current amount determined on the basis of the operation result in the control device 20. Furthermore, the control unit may be provided in each joint unit and may control drive of each joint unit.

The arm unit 120 is configured as a multilink structure including a plurality of joint units and a plurality of links, for example, and drive of the arm unit 120 is controlled by the control of the arm control unit 110. The arm unit 120 corresponds to the arm unit 5031 illustrated in FIG. 1. The arm unit 120 includes the joint unit 130 and the imaging unit 140. Note that, since functions and configurations of the plurality of joint units included in the arm unit 120 are similar to one another, FIG. 6 illustrates a configuration of one joint unit 130 as a representative of the plurality of joint units.

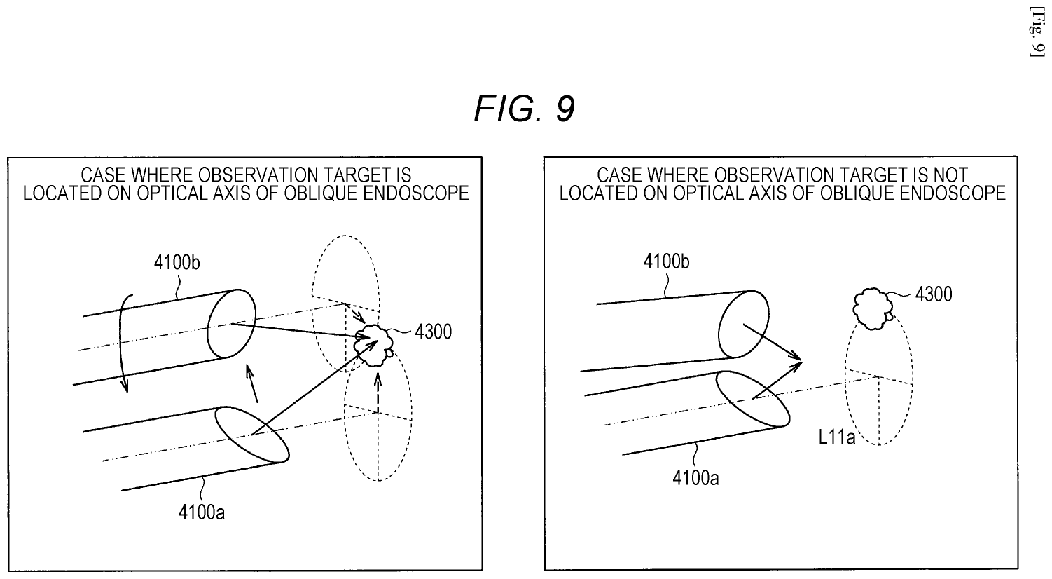

The joint unit 130 rotatably connects the links with each other in the arm unit 120, and drives the arm unit 120 as rotational drive of the joint unit 130 is controlled by the control of the arm control unit 110. The joint unit 130 corresponds to the joint units 421a to 421f illustrated in FIG. 8. Furthermore, the joint unit 130 includes an actuator, and the configuration of the actuator is similar to the configuration illustrated in FIGS. 3 and 9, for example.

The joint unit 130 includes a joint drive unit 131 and a joint state detection unit 132.

The joint drive unit 131 is a drive mechanism in the actuator of the joint unit 130, and the joint unit 130 is rotationally driven as the joint drive unit 131 is driven. The drive of the joint drive unit 131 is controlled by the drive control unit 111. For example, the joint drive unit 131 is a configuration corresponding to a driver for driving the actuators respectively provided in the joint units 5033a to 5033c illustrated in FIG. 1, and drive of the joint drive unit 131 being driven corresponds to the driver driving the actuators with the current amount according to a command from the drive control unit 111.

The joint state detection unit 132 detects a state of the joint unit 130. Here, the state of the joint unit 130 may mean a state of motion of the joint unit 130. For example, the state of the joint unit 130 includes information of the rotation angle, rotation angular speed, rotation angular acceleration, generated torque of the joint unit 130, or the like, which indicates a state of rotation of the joint unit 130. In the present embodiment, the joint state detection unit 132 has a rotation angle detection unit 133 that detects the rotation angle of the joint unit 130 and a torque detection unit 134 that detects the generated torque and external torque of the joint unit 130. The joint state detection unit 132 transmits the detected state of the joint unit 130 to the control device 20.

The imaging unit 140 is an example of the distal end unit provided at the distal end of the arm unit 120, and acquires an image of a capture target. A specific example of the imaging unit 140 includes the endoscope device 423 illustrated in FIG. 3. Specifically, the imaging unit 140 is a camera or the like that can capture the capture target in the form of a moving image or a still image. More specifically, the imaging unit 140 includes a plurality of light receiving elements arranged in a two dimensional manner, and can obtain an image signal representing an image of the capture target by photoelectric conversion in the light receiving elements. The imaging unit 140 transmits the acquired image signal to the display device 30.

Note that, as in the case of the support arm device 400 illustrated in FIG. 3, where the endoscope device 423 is provided at the distal end of the arm unit 420, the imaging unit 140 is actually provided at the distal end of the arm unit 120 in the support arm device 10. FIG. 6 illustrates a state in which the imaging unit 140 is provided at a distal end of a final link via the plurality of joint units 130 and the plurality of links by schematically illustrating a link between the joint unit 130 and the imaging unit 140.

Note that, in the present embodiment, various medical instruments can be connected to the distal end of the arm unit 120 as the distal end unit. Examples of the medical instruments include various treatment instruments such as a scalpel and forceps, and various units used in treatment, such as a unit of various detection devices such as probes of an ultrasonic examination device. Furthermore, in the present embodiment, the imaging unit 140 illustrated in FIG. 6 or a unit having an imaging function such as an endoscope or a microscope may also be included in the medical instruments. Thus, the support arm device 10 according to the present embodiment can be said to be a medical support arm device provided with medical instruments. Similarly, the medical arm system 1 according to the present embodiment can be said to be a medical arm system. Note that the support arm device 10 illustrated in FIG. 6 can also be said to be a video endoscope support arm device provided with a unit having an imaging function as the distal end unit.

The function and configuration of the support arm device 10 have been described above. Next, a function and a configuration of the control device 20 will be described. Referring to FIG. 6, the control device 20 includes an input unit 210, a storage unit 220, and a control unit 230.

The control unit 230 integrally controls the control device 20 and performs various operations for controlling the drive of the arm unit 120 in the support arm device 10. Specifically, to control the drive of the arm unit 120 of the support arm device 10, the control unit 230 performs various operations in known whole body coordination control and ideal joint control, for example.

The control unit 230 includes a whole body coordination control unit 240 and an ideal joint control unit 250.

The whole body coordination control unit 240 performs various operations regarding the whole body coordination control using the generalized inverse dynamics. In the present embodiment, the whole body coordination control unit 240 acquires a state (arm state) of the arm unit 120 on the basis of the state of the joint unit 130 detected by the joint state detection unit 132. Furthermore, the whole body coordination control unit 240 calculates a control value for the whole body coordination control of the arm unit 120 in an operation space, using the generalized inverse dynamics, on the basis of the arm state, and a motion purpose and a constraint condition of the arm unit 120. Note that the operation space is a space for describing the relationship between the force acting on the arm unit 120 and the acceleration generated in the arm unit 120, for example. In this embodiment, the whole body coordination control unit 240 controls the arm unit.

The whole body coordination control unit 240 includes an arm state unit 241, an arithmetic condition setting unit 242, a virtual force calculation unit 243, and a real force calculation unit 244.

The arm state unit 241 acquires the state of the arm unit 120 on the basis of the state of the joint unit 130 detected by the joint state detection unit 132. Here, the arm state may mean the state of motion of the arm unit 120. For example, the arm state includes information such as the position, speed, acceleration, and force of the arm unit 120. As described above, the joint state detection unit 132 acquires, as the state of the joint unit 130, the information of the rotation angle, rotation angular speed, rotation angular acceleration, generated torque in each joint unit 130, and the like. Furthermore, although to be described below, the storage unit 220 stores various types of information to be processed by the control device 20. In the present embodiment, the storage unit 220 may store various types of information (arm state information) regarding the arm unit 120, for example, information regarding the configuration of the arm unit 120, in other words, the number of joint units 130 and links configuring the arm unit 120, connection situations between the links and the joint units 130, and lengths of the links, and the like. The arm state unit 241 can acquire the arm state information from the storage unit 220. Therefore, the arm state unit 241 can acquire, as the arm state, information such as the positions (coordinates) in the space of the plurality of joint units 130, the plurality of links, and the imaging unit 140 (in other words, the shape of the arm unit 120 and the position and posture of the imaging unit 140), and the forces acting on the joint units 130, the links, and the imaging unit 140, on the basis of the state and the arm information of the joint units 130.

In other words, the arm state unit 241 can acquire information regarding position and posture of a point of action set using at least a part of the arm unit 120 as a base point as the arm state. As a specific example, the arm state unit 241 can recognize the position of the point of action as a relative position relative to the part of the arm unit 120 on the basis of the information of the position, posture, shape of the joint units 130 and the links configuring the arm unit 120. Furthermore, the point of action may be set at a position corresponding to a part (for example, a distal end or the like) of the distal end unit by taking into account the position, posture, shape of the distal end unit (for example, the imaging unit 140) held by the arm unit 120. Furthermore, the position where the point of action is set is not limited to only a part of the distal end unit or a part of the arm unit 120. For example, in a state where the distal end unit is not supported by the arm unit 120, the point of action may be set at a position (space) corresponding to the distal end unit in a case where the distal end unit is supported by the arm unit 120. Note that the information regarding the position and posture of the point of action acquired as described above (in other words, the information acquired as the arm state) corresponds to an example of “arm state information”.

Then, the arm state unit 241 transmits the acquired arm information to the arithmetic condition setting unit 242.

The arithmetic condition setting unit 242 sets operation conditions in an operation regarding the whole body coordination control using the generalized inverse dynamics. Here, the operation condition may be a motion purpose and a constraint condition. The motion purpose may be various types of information regarding the motion of the arm unit 120. Specifically, the motion purpose may be target values of the position and posture (coordinates), speed, acceleration, force of the imaging unit 140, or target values of the positions and postures (coordinates), speeds, accelerations, forces of the plurality of joint units 130 and the plurality of links of the arm unit 120. Furthermore, the constraint condition may be various types of information that restricts (restrains) the motion of the arm unit 120. Specifically, the constraint condition may include coordinates of a region where each configuration component of the arm unit cannot move, an unmovable speed, a value of acceleration, a value of an force which cannot be generated, and the like. Furthermore, restriction ranges of various physical quantities under the constraint condition may be set according to inability to structurally realizing the arm unit 120 or may be appropriately set by the user. Furthermore, the arithmetic condition setting unit 242 includes a physical model for the structure of the arm unit 120 (in which, for example, the number and lengths of the links configuring the arm unit 120, the connection states of the links via the joint units 130, the movable ranges of the joint units 130, and the like are modeled), and may set a motion condition and the constraint condition by generating a control model in which the desired motion condition and constraint condition are reflected in the physical model.

Furthermore, the arithmetic condition setting unit 242 may set the motion condition and the constraint condition on the basis of information according to a detection result by a detector such as various sensors. As a specific example, the arithmetic condition setting unit 242 may set the motion condition and the constraint condition taking into account information (for example, information regarding a space around a unit) acquired by the unit (for example, the imaging unit 140) supported by the arm unit 120. As a more specific example, the arithmetic condition setting unit 242 may estimate the position and posture of the point of action (in other words, a self-position of the point of action) on the basis of the arm information, and generate or update an environment map regarding a space around the point of action (for example, a map regarding a three-dimensional space of a body cavity or a surgical field) on the basis of a result of the estimation and the information acquired by the above unit. An example of a technology regarding the estimation of the self-position and the generation of the environment map includes a technology called simultaneous localization and mapping (SLAM). Then, the arithmetic condition setting unit 242 may set the motion condition and the constraint condition on the basis of the self-position of the point of action and the environment map. Note that the above unit (sensor unit) in this case corresponds to an example of an “acquisition unit”, and the information (sensor information) acquired by the unit corresponds to an example of “environment information”. Furthermore, the environment map corresponds to an example of “mapping information”.

In the present embodiment, appropriate setting of the motion purpose and the constraint condition enables the arm unit 120 to perform a desired operation. For example, not only can the imaging unit 140 be moved to a target position by setting a target value of the position of the imaging unit 140 as the motion purpose but also the arm unit 120 can be driven by providing a constraint of movement by the constraint condition to prevent the arm unit 120 from intruding into a predetermined region in the space. Furthermore, by use of the environment map, for example, a constraint condition is set according to a situation around the imaging unit 140, such as avoiding a contact between the imaging unit 140 with another object (for example, an organ or the like), and the arm unit 120 can be driven providing movement constraint by the constraint condition.

A specific example of the motion purpose includes, for example, a pivot operation (for example, a turning operation with an axis of a cone serving as a pivot axis, in which the imaging unit 140 moves in a conical surface setting an operation site as a top) in a state where the capture direction of the imaging unit 140 is fixed to the operation site. Furthermore, in the pivot operation, the turning operation may be performed in a state where the distance between the imaging unit 140 and a point corresponding to the top of the cone is kept constant. By performing such a pivot operation, an observation site can be observed from an equal distance and at different angles, whereby the convenience of the user who performs surgery can be improved.

Furthermore, as another specific example, the motion purpose may be content to control the generated torque in each joint unit 130. Specifically, the motion purpose may be a power assist operation to control the state of the joint unit 130 to cancel the gravity acting on the arm unit 120, and further control the state of the joint unit 130 to support the movement of the arm unit 120 in a direction of a force provided from the outside. More specifically, in the power assist operation, the drive of each joint unit 130 is controlled to cause each joint unit 130 to generate a generated torque that cancels the external torque due to the gravity in each joint unit 130 of the arm unit 120, whereby the position and posture of the arm unit 120 are held in a predetermined state. In a case where an external torque is further added from the outside (for example, from the user) in the aforementioned state, the drive of each joint unit 130 is controlled to cause each joint unit 130 to generate a generated torque in the same direction as the added external torque. By performing such a power assist operation, the user can move the arm unit 120 with a smaller force in a case where the user manually moves the arm unit 120. Therefore, a feeling as if the user moved the arm unit 120 under weightlessness can be provided to the user. Furthermore, the above-described pivot operation and the power assist operation can be combined.

Here, in the present embodiment, the motion purpose may mean an operation (motion) of the arm unit 120 realized by the whole body coordination control or may mean an instantaneous motion purpose in the operation (in other words, a target value in the motion purpose). For example, in the above-described pivot operation, the imaging unit 140 performing the pivot operation itself is the motion purpose. In the act of performing the pivot operation, values of the position, speed of the imaging unit 140 in a conical surface in the pivot operation are set as the instantaneous motion purpose (the target values in the motion purpose). Furthermore, in the above-described power assist operation, for example, performing the power assist operation to support the movement of the arm unit 120 in the direction of the force applied from the outside itself is the motion purpose. In the act of performing the power assist operation, the value of the generated torque in the same direction as the external torque applied to each joint unit 130 is set as the instantaneous motion purpose (the target value in the motion purpose). The motion purpose in the present embodiment is a concept including both the instantaneous motion purpose (for example, the target values of the positions, speeds, forces of the configuration members of the arm unit 120 at a certain time) and the operations of the configuration members of the arm unit 120 realized over time as a result of the instantaneous motion purpose having been continuously achieved. The instantaneous motion purpose is set each time in each step in an operation for the whole body coordination control in the whole body coordination control unit 240, and the operation is repeatedly performed, so that the desired motion purpose is finally achieved.

Note that, in the present embodiment, the viscous drag coefficient in a rotation motion of each joint unit 130 may be appropriately set when the motion purpose is set. As described above, the joint unit 130 according to the present embodiment is configured to be able to appropriately adjust the viscous drag coefficient in the rotation motion of the actuator. Therefore, by setting the viscous drag coefficient in the rotation motion of each joint unit 130 when setting the motion purpose, an easily rotatable state or a less easily rotatable state can be realized for the force applied from the outside, for example. For example, in the above-descried power assist operation, when the viscous drag coefficient in the joint unit 130 is set to be small, a force required by the user to move the arm unit 120 can be made small, and a weightless feeling provided to the user can be promoted. As described above, the viscous drag coefficient in the rotation motion of each joint unit 130 may be appropriately set according to the content of the motion purpose.

In the present embodiment, the storage unit 220 may store parameters regarding the operation conditions such as the motion purpose and the constraint condition used in the operation regarding the whole body coordination control. The arithmetic condition setting unit 242 can set the constraint condition stored in the storage unit 220 as the constraint condition used for the operation of the whole body coordination control.

Furthermore, in the present embodiment, the arithmetic condition setting unit 242 can set the motion purpose by a plurality of methods. For example, the arithmetic condition setting unit 242 may set the motion purpose on the basis of the arm state transmitted from the arm state unit 241. As described above, the arm state includes information of the position of the arm unit 120 and information of the force acting on the arm unit 120. Therefore, for example, in a case where the user is trying to manually move the arm unit 120, information regarding how the user is moving the arm unit 120 is also acquired by the arm state unit 241 as the arm state. Therefore, the arithmetic condition setting unit 242 can set the position, speed, force to/at/with which the user has moved the arm unit 120, as the instantaneous motion purpose, on the basis of the acquired arm state. By thus setting the motion purpose, the drive of the arm unit 120 is controlled to follow and support the movement of the arm unit 120 by the user.

Furthermore, for example, the arithmetic condition setting unit 242 may set the motion purpose on the basis of an instruction input from the input unit 210 by the user. Although to be described below, the input unit 210 is an input interface for the user to input information, commands regarding the drive control of the support arm device 10, to the control device 20. In the present embodiment, the motion purpose may be set on the basis of an operation input from the input unit 210 by the user. Specifically, the input unit 210 has, for example, operation unit operated by the user, such as a lever and a pedal. The positions, speeds of the configuration members of the arm unit 120 may be set as the instantaneous motion purpose by the arithmetic condition setting unit 242 in response to an operation of the lever, pedalor the like.

Moreover, for example, the arithmetic condition setting unit 242 may set the motion purpose stored in the storage unit 220 as the motion purpose used for the operation of the whole body coordination control. For example, in the case of the motion purpose that the imaging unit 140 stands still at a predetermined point in the space, coordinates of the predetermined point can be set in advance as the motion purpose. Furthermore, for example, in the case of the motion purpose that the imaging unit 140 moves on a predetermined trajectory in the space, coordinates of each point representing the predetermined trajectory can be set in advance as the motion purpose. As described above, in a case where the motion purpose can be set in advance, the motion purpose may be stored in the storage unit 220 in advance. Furthermore, in the case of the above-described pivot operation, for example, the motion purpose is limited to a motion purpose setting the position, speed, and the like in the conical surface as the target values. In the case of the power assist operation, the motion purpose is limited to a motion purpose setting the force as the target value. In the case where the motion purpose such as the pivot operation or the power assist operation is set in advance in this way, information regarding ranges, types and the like of the target values settable as the instantaneous motion purpose in such a motion purpose may be stored in the storage unit 220. The arithmetic condition setting unit 242 can also set the various types of information regarding such a motion purpose as the motion purpose.

Note that by which method the arithmetic condition setting unit 242 sets the motion purpose may be able to be appropriately set by the user according to the application of the support arm device 10 or the like. Furthermore, the arithmetic condition setting unit 242 may set the motion purpose and the constraint condition by appropriately combining the above-described methods. Note that a priority of the motion purpose may be set in the constraint condition stored in the storage unit 220, or in a case where there is a plurality of motion purposes different from one another, the arithmetic condition setting unit 242 may set the motion purpose according to the priority of the constraint condition. The arithmetic condition setting unit 242 transmits the arm state and the set motion purpose and constraint condition to the virtual force calculation unit 243.

The virtual force calculation unit 243 calculates a virtual force in the operation regarding the whole body coordination control using the generalized inverse dynamics. Note that, as for the virtual force calculation processing, application of a well-known technology regarding whole body coordination control using the generalized inverse dynamics is possible. Therefore, detailed description is omitted. The virtual force calculation unit 243 transmits the calculated virtual force to the real force calculation unit 244.

The real force calculation unit 244 calculates a real force in the operation regarding the whole body coordination control using the generalized inverse dynamics. Note that, as for the real force calculation processing, application of a well-known technology regarding whole body coordination control using the generalized inverse dynamics is possible. Therefore, detailed description is omitted. The real force calculation unit 244 transmits the calculated real force (generated torque) Tₐ to the ideal joint control unit 250. Note that, in the present embodiment, the generated torque Tₐ calculated by the real force calculation unit 244 is also referred to as a control value or a control torque value in the sense of a control value of the joint unit 130 in the whole body coordination control.

The ideal joint control unit 250 performs various operations regarding the ideal joint control that realizes an ideal response based on a theoretical model. In the present embodiment, the ideal joint control unit 250 corrects the influence of disturbance for the generated torque Tₐ calculated by the real force calculation unit 244 to calculate a torque command value T realizing an ideal response of the arm unit 120. Note that, as for the operation processing performed by the ideal joint control unit 250, application of a known technology regarding ideal joint control is possible. Therefore, detailed description is omitted.

The ideal joint control unit 250 includes a disturbance estimation unit 251 and a command value calculation unit 252.

The disturbance estimation unit 251 calculates a disturbance estimation value τ_{d} on the basis of the torque command value T and the rotation angular speed calculated from the rotation angle q detected by the rotation angle detection unit 133. Note that the torque command value T mentioned here is a command value that represents the generated torque in the arm unit 120 to be finally transmitted to the support arm device 10.

The command value calculation unit 252 calculates the torque command value T that is a command value representing the torque to be generated in the arm unit 120 and finally transmitted to the support arm device 10, using the disturbance estimation value Td calculated by the disturbance estimation unit 251. Specifically, the command value calculation unit 252 adds the disturbance estimation value τ_{d} calculated by the disturbance estimation unit 251 to a torque target value τ^{ref} to calculate the torque command value τ. Note that the torque target value τ^{ref} can be calculated, for example, from an ideal model expressed as an equation of motion of a second-order lag system in known ideal joint control. For example, in a case where the disturbance estimation value τ_{d} is not calculated, the torque command value τ becomes the torque target value τ^{ref}.

As described above, in the ideal joint control unit 250, the information is repeatedly exchanged between the disturbance estimation unit 251 and the command value calculation unit 252, so that the series of processing regarding the ideal joint control (in other words, various operations regarding the ideal joint control) is performed. The ideal joint control unit 250 transmits the calculated torque command value τ to the drive control unit 111 of the support arm device 10. The drive control unit 111 performs control to supply the current amount corresponding to the transmitted torque command value τ to the motor in the actuator of the joint unit 130, thereby controlling the number of rotations of the motor and controlling the rotation angle and the generated torque in the joint unit 130.

In the medical arm system 1 according to the present embodiment, the drive control of the arm unit 120 in the support arm device 10 is continuously performed during work using the arm unit 120, so the above-described processing in the support arm device 10 and the control device 20 is repeatedly performed. In other words, the state of the joint unit 130 is detected by the joint state detection unit 132 of the support arm device 10 and transmitted to the control device 20. The control device 20 performs various operations regarding the whole body coordination control and the ideal joint control for controlling the drive of the arm unit 120 on the basis of the state of the joint unit 130, and the motion purpose and the constraint condition, and transmits the torque command value τ as the operation result to the support arm device 10. The support arm device 10 controls the drive of the arm unit 120 on the basis of the torque command value τ, and the state of the joint unit 130 during or after the drive is detected by the joint state detection unit 132 again.

Description about other configurations included in the control device 20 will be continued.

The input unit 210 is an input interface for the user to input information, commands regarding the drive control of the support arm device 10 to the control device 20. In the present embodiment, the drive of the arm unit 120 of the support arm device 10 may be controlled on the basis of the operation input from the input unit 210 by the user, and the position and posture of the imaging unit 140 may be controlled. Specifically, as described above, instruction information regarding the instruction of the drive of the arm input from the input unit 210 by the user is input to the arithmetic condition setting unit 242, so that the arithmetic condition setting unit 242 may set the motion purpose in the whole body coordination control on the basis of the instruction information. The whole body coordination control is performed using the motion purpose based on the instruction information input by the user as described above, so that the drive of the arm unit 120 according to the operation input of the user is realized.

Specifically, the input unit 210 includes operation unit operated by the user, such as a mouse, a keyboard, a touch panel, a button, a switch, a lever, and a pedal, for example. For example, in a case where the input unit 210 has a pedal, the user can control the drive of the arm unit 120 by operating the pedal with the foot. Therefore, even in a case where the user is performing treatment using both hands on the operation site of the patient, the user can adjust the position and posture of the imaging unit 140, in other words, the user can adjust a capture position and a capture angle of the operation site, by the operation of the pedal with the foot.

The storage unit 220 stores various types of information processed by the control device 20. In the present embodiment, the storage unit 220 can store various parameters used in the operation regarding the whole body coordination control and the ideal joint control performed by the control unit 230. For example, the storage unit 220 may store the motion purpose and the constraint condition used in the operation regarding the whole body coordination control by the whole body coordination control unit 240. The motion purpose stored in the storage unit 220 may be, as described above, a motion purpose that can be set in advance, such as, for example, the imaging unit 140 standing still at a predetermined point in the space. Furthermore, the constraint conditions may be set in advance by the user and stored in the storage unit 220 according to a geometric configuration of the arm unit 120, the application of the support arm device 10, and the like. Furthermore, the storage unit 220 may also store various types of information regarding the arm unit 120 used when the arm state unit 241 acquires the arm state. Moreover, the storage unit 220 may store the operation result, various numerical values calculated in the operation process in the operation regarding the whole body coordination control and the ideal joint control by the control unit 230. As described above, the storage unit 220 may store any parameters regarding the various types of processing performed by the control unit 230, and the control unit 230 can performs various types of processing while mutually exchanging information with the storage unit 220.

The function and configuration of the control device 20 have been described above. Note that the control device 20 according to the present embodiment can be configured by, for example, various information processing devices (arithmetic processing devices) such as a personal computer (PC) and a server. Next, a function and a configuration of the display device 30 will be described.

The display device 30 displays the information on the display screen in various formats such as texts and images to visually notify the user of various types of information. In the present embodiment, the display device 30 displays the image captured by the imaging unit 140 of the support arm device 10 on the display screen. Specifically, the display device 30 has functions and configurations of an image signal processing unit (not illustrated) that applies various types of image processing to an image signal acquired by the imaging unit 140, a display control unit (not illustrated) that performs control to display an image based on the processed image signal on the display screen, and the like. Note that the display device 30 may have various functions and configurations that a display device generally has, in addition to the above-described functions and configurations. The display device 30 corresponds to, for example, the display device 5041 illustrated in FIG. 1.

The functions and configurations of the support arm device 10, the control device 20, and the display device 30 according to the present embodiment have been described above with reference to FIG. 6. Each of the above-described constituent elements may be configured using general-purpose members or circuit, or may be configured by hardware specialized for the function of each constituent element. Furthermore, all the functions of the configuration elements may be performed by a CPU or the like. Therefore, the configuration to be used can be changed as appropriate according to the technical level of the time of carrying out the present embodiment.

As described above, according to the present embodiment, the arm unit 120 that is the multilink structure in the support arm device 10 has at least six degrees or more of freedom, and the drive of each of the plurality of joint units 130 configuring the arm unit 120 is controlled by the drive control unit 111. Then, a medical instrument is provided at the distal end of the arm unit 120. The drive of each of the joint units 130 is controlled as described above, so that the drive control of the arm unit 120 with a higher degree of freedom is realized, and the support arm device 10 with higher operability for the user is realized.

More specifically, according to the present embodiment, the joint state detection unit 132 detects the state of the joint unit 130 in the support arm device 10. Then, the control device 20 performs various operations regarding the whole body coordination control using the generalized inverse dynamics for controlling the drive of the arm unit 120 on the basis of the state of the joint unit 130, and the motion purpose and the constraint condition, and calculates the torque command value T as the operation result. Moreover, the support arm device 10 controls the drive of the arm unit 120 on the basis of the torque command value τ. As described above, in the present embodiment, the drive of the arm unit 120 is controlled by the whole body coordination control using the generalized inverse dynamics. Therefore, the drive control of the arm unit 120 by force control is realized, and a support arm device with higher operability for the user is realized. Furthermore, in the present embodiment, control to realize various motion purposes for further improving the convenience of the user, such as the pivot operation and the power assist operation, for example, is possible in the whole body coordination control. Moreover, in the present embodiment, various driving unit are realized, such as manually moving the arm unit 120, and moving the arm unit 120 by the operation input from a pedal, for example. Therefore, further improvement of the convenience for the user is realized.

Furthermore, in the present embodiment, the ideal joint control is applied together with the whole body coordination control to the drive control of the arm unit 120. In the ideal joint control, the disturbance components such as friction and inertia inside the joint unit 130 are estimated, and the feedforward control using the estimated disturbance components is performed. Therefore, even in a case where there is a disturbance component such as friction, an ideal response can be realized for the drive of the joint unit 130. Therefore, in the drive control of the arm unit 120, highly accurate response and high positioning accuracy and stability with less influence of vibration and the like are realized.

Moreover, in the present embodiment, each of the plurality of joint units 130 configuring the arm unit 120 has a configuration adapted to the ideal joint control, for example, as illustrated in FIG. 3, and the rotation angle, generated torque, and viscous drag coefficient in each joint unit 130 can be controlled with the current value. As described above, the drive of each joint unit 130 is controlled with the current value, and the drive of each joint unit 130 is controlled while grasping the state of the entire arm unit 120 by the whole body coordination control. Therefore, counterbalance is unnecessary and downsizing of the support arm device 10 is realized.

Note that an example of a case where the arm unit 120 is configured as a multilink structure has been described. However, the example does not necessarily limit the configuration of the medical arm system 1 according to an embodiment of the present disclosure. In other words, the configuration of the arm unit 120 is not particularly limited as long as the position and posture of the arm unit 120 are recognized and the operation of the arm unit 120 can be controlled on the basis of the technology regarding the whole body coordination control and the ideal joint control according to the result of the recognition. As a specific example, a portion corresponding to the arm unit 120 may be configured as a flexible member in which at least a part is bendable like a distal end portion of a so-called flexible endoscope, thereby controlling the position and posture of the medical instrument provided at the distal end. Notably, whilst the whole body coordination control unit 240 of the control device has been described herein as calculating the control command value for the whole body coordination control, for example using inverse dynamics, this is a non-limiting example. Rather, any suitable technique for control of some or all of the multilink structure (or any other form of articulated medical arm) may be considered.

### 5. Control of Arm

### 5.1. Overview

Next, control of an arm in the medical arm system according to an embodiment of the present disclosure will be described. In the medical arm system 1 according to the present embodiment, information regarding a space around a set point of action (for example, a space around a unit supported by the arm unit 120 (for example, the distal end unit such as an endoscope) (hereinafter the information is also referred to as “environment map” for convenience) is generated or updated using the information acquired by the unit and the information regarding the position and posture of the arm unit 120 (arm information). With such a configuration, an environment map of a space in a body cavity of a patient can also be generated, for example. In the medical arm system according to the present embodiment, the environment map is used for control of the operation of the arm unit 120 (for example, control of the position and posture, feedback of a reaction force against an external force, or the like) under such a configuration.

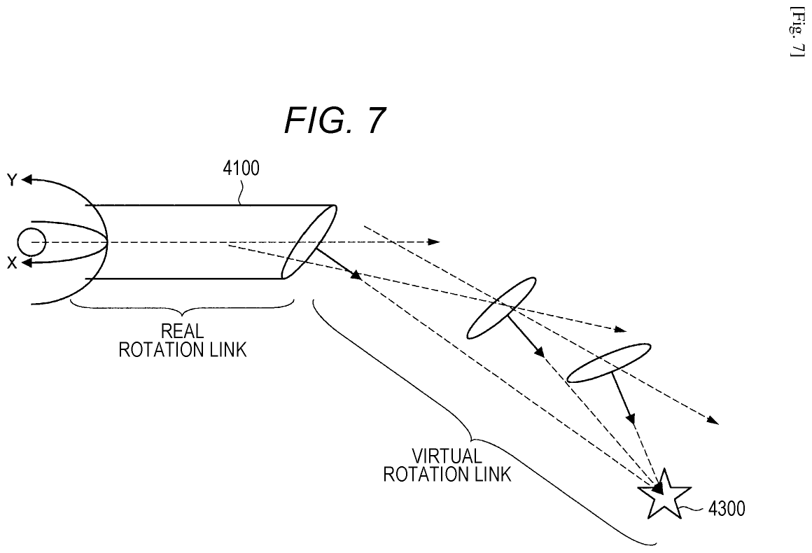

Here, to make the characteristics of the arm control in the medical arm system according to the present embodiment more understandable, an example of the arm control in a case of performing an observation using an oblique endoscope will be described with reference to FIGS. 7 and 8. FIGS. 7 and 8 are explanatory diagrams for describing an overview of an example of the arm control in the case of performing an observation using an oblique endoscope.

For example, in the example illustrated in FIGS. 7 and 8, a hard endoscope axis C2 in the example illustrated in the right diagram in FIG. 5 is set as an axis of a real link (real rotation link), and an oblique endoscope optical axis C1 is set as an axis of a virtual link (virtual rotation link). An oblique endoscope unit is modeled as a plurality of interlocking links and the arm control is performed under such setting, so that control for maintaining hand-eye coordination of an operator is possible, as illustrated in FIGS. 7 and 8.

Specifically, FIG. 7 is a diagram for describing update of a virtual rotation link in consideration of a zoom operation of an oblique endoscope. FIG. 7 illustrates an oblique endoscope 4100 and an observation target 4300. For example, as illustrated in FIG. 7, in a case where the zoom operation is performed, control to capture the observation target 4300 in the center of the camera becomes possible by changing the distance and direction of the virtual rotation link (making the distance of the virtual rotation link short and largely inclining the direction of the virtual rotation link with respect to a scope axis in a case of an enlargement operation as illustrated in FIG. 7).

Furthermore, FIG. 8 is a diagram for describing update of a virtual rotation link in consideration of a rotation operation of the oblique endoscope. FIG. 8 illustrates the oblique endoscope 4100 and the observation target 4300. As illustrated in FIG. 8, in a case where the rotation operation is performed, control to capture the observation target 4300 in the center of the camera becomes possible by making the distance of the virtual rotation link constant.

Next, an example of technical problems that may be caused in a case where use of the information of an inside of a patient (for example, the environment map) is difficult will be described focusing on a case of performing an observation using an oblique endoscope, with reference to FIG. 9. FIG. 9 is an explanatory diagram for describing an example of technical problems in a case of performing an observation using an oblique endoscope, and illustrates an example of a case of observing the observation target 4300 from different directions by performing a rotation operation, as in the example described with reference to FIG. 8. FIG. 9 schematically illustrates respective positions 4100a and 4100b of the oblique endoscope 4100 in a case of observing the observation target 4300 from different directions from each other.

For example, in a case of maintaining the state where the observation target 4300 is captured in the center of the camera under the situation where the observation target 4300 is observed from different directions, it is desirable to control the position and posture of the oblique endoscope 4100 such that the observation target 4300 (in particular, a point of interest of the observation target 4300) is located on an optical axis of the oblique endoscope 4100. As a specific example, the left diagram in FIG. 9 schematically illustrates a situation in which the state where the observation target 4300 is located on the optical axis of the oblique endoscope 4100 is maintained even in a case of changing the position and posture of the oblique endoscope 4100.

In contrast, the right diagram in FIG. 9 schematically illustrates a situation in which the observation target 4300 is not located on the optical axis of the oblique endoscope 4100 in the case where the oblique endoscope 4100 is located at the position 4100b. Under such a situation, maintaining the state in which the observation target 4300 is captured in the center of the camera is difficult when the position and posture of the oblique endoscope 4100 is changed. In other words, the observation target 4300 is presented at a position distant from a center of a screen, and a situation where the observation target 4300 is not presented on the screen (in other words, a situation where the observation target 4300 is located outside the screen) can be assumed, accordingly. In view of such a situation, it is more desirable to three-dimensionally recognize the position and posture of the observation target 4300.

Furthermore, as in the example described with reference to FIG. 7, in a case where only insertion/removal operation in the longitudinal direction of the oblique endoscope 4100 has been performed under a situation where the zoom operation by the insertion/removal operation of the oblique endoscope 4100 (in other words, the endoscope unit) is performed, the observation target 4300 may not be located on the optical axis of the oblique endoscope 4100. In other words, even under the situation where the zoom operation is performed, to maintain the state where the observation target 4300 is captured in the center of the camera, it is desirable to control the position and posture of the oblique endoscope 4100 to maintain the state where the observation target 4300 is located on the optical axis of the oblique endoscope 4100.

Note that, according to the medical arm system 1 of the present disclosure, the position and posture of the endoscope device (oblique endoscope 4100) supported by the arm unit 120 can be recognized as the arm information according to the state of the arm unit 120. In other words, three-dimensional position and posture of the unit (in other words, the point of action) supported by the arm unit 120 can be recognized on the basis of mechanical information (a rotary encoder or a linear encoder) and dynamical information (a mass, inertia, a center of gravity position, a torque sensor, or a force sensor) of the arm unit 120 itself. However, it is difficult to recognize an external environment of the arm unit 120 only from the above-described mechanical information and dynamical information, in some cases.

In view of such a situation, the present disclosure proposes a technology for enabling control the operation of the arm unit 120 in a more favorable form according to a surrounding situation. Specifically, the medical arm system 1 according to an embodiment of the present disclosure generates or updates the environment map regarding the external environment (in particular, the space around the point of action) of the arm unit 120 on the basis of the information acquired from the imaging unit (for example, the endoscope device or the like) supported by the arm unit 120 or various sensors. The medical arm system 1 more accurately recognizes the position and posture of the observation target 4300 on the basis of the environment map and uses the recognition result for the control (for example, position control, speed control, force control, and the like) of the arm unit 120.

### 5.2. Environment Map Generation Method

Next, an example of a method regarding generation or update of the environment map regarding the external environment of the arm unit 120 will be described below.

(Method of Using Captured Image)

The environment map can be generated or updated by reconstructing a three-dimensional space using an image (still image or moving image) captured by the imaging unit (image sensor) such as the endoscope device supported by the arm unit 120 as the distal end unit. A specific example includes a method of generating or updating the environment map using characteristic points extracted from captured images. In this case, the characteristic points (for example, vertexes, edges, and the like of an object) are extracted by applying an image analysis to the captured images, and the three-dimensional space is reconstructed by an application of triangulation from correspondence among the characteristic points extracted from a plurality of captured images. In a case where the imaging unit (endoscope device) captures 2D images, which are widely used, the three-dimensional space can be reconstructed by using a plurality of images captured from different positions. Furthermore, a plurality of (for example, two) images can be captured at the same time in a case where the imaging unit is configured as a stereo camera. Therefore, the three-dimensional space can be reconstructed on the basis of the correspondence between the characteristic points extracted from the images between the plurality of images.

Furthermore, in a case of using an endoscope image as the captured image, the three-dimensional space can be reconstructed without additionally providing a sensor to the arm unit 120 that supports the endoscope device, and the environment map can be generated or updated on the basis of a result of the reconstruction.

Note that in a case of reconstructing the three-dimensional space using the captured image, it may be difficult to specify a unit (for example, SI unit system or the like) of a real space from the captured image. In such a case, the unit can also be specified by combining the captured image used to reconstruct the three-dimensional space and the mechanical information (kinematics) of the arm unit 120 at the time of capturing the captured image.

The position and posture of the arm unit and the position and posture based on the analysis result of the captured image can be modeled as described in (Expression 1) and (Expression 2) below.

$$\begin{matrix}[ Math . 1 ] & \\ {S}_{c \to r} {p}_{c} + {t}_{c \to r} = {p}_{r} & ( Expression 1 ) \\ {R}_{c \to r} \cdot {R}_{c} = {R}_{r} & ( Expression 2 )\end{matrix}$$

In the above (Expression 1), p_{c} represents the position (three-dimensional vector) of the characteristic point in a coordinate system of the captured image. In contrast, pᵣ represents the position (three-dimensional vector) of the characteristic point in a coordinate system of the arm unit. Furthermore, R_{c} represents the posture (3×3 matrix) of the characteristic point in the coordinate system of the captured image. In contrast, Rᵣ represents the posture (3×3 matrix) of the characteristic point in the coordinate system of the arm unit. Furthermore, S_{c→r} represents a scaling coefficient (scalar value) between the coordinate system of the captured image and the coordinate system of the arm unit. Furthermore, t_{c→r} represents an offset (three-dimensional vector) for associating (for example, substantially matching) the coordinate system of the captured image with the coordinate system of the arm unit. Furthermore, R_{c→r} represents a rotation matrix (3×3 matrix) for associating (for example, substantially matching) the coordinate system of the captured image with the coordinate system of the arm unit. In other words, if p_{c} and pᵣ, and R_{c} and Rᵣ are known for two or more characteristic points on the basis of the above (Expression 1) and (Expression 2), S_{c→r}, t_{c→r}, and R_{c→r} can be calculated.

Furthermore, as another example, the environment map may be generated or updated by reconstructing the three-dimensional space on the basis of information regarding color (in other words, a color space) extracted from the captured image. Note that the information used as the color space in this case is not specifically limited. As a specific example, a model of an RGB colorimetric system may be applied or an HSV model may be applied.

(Method of Using Distance Measurement Sensor)

The environment map can be generated or updated by reconstructing the three-dimensional space using a measurement result of a distance (depth) between an object in the real space and a distance measurement sensor supported by a part of the arm unit 120. A specific example of the distance measurement sensor includes a time of flight (ToF) sensor. The ToF sensor measures a time from when the light is projected from the light source to when reflected light reflected by the object is detected, thereby calculating the distance to the object on the basis of the measurement result. In this case, for example, since distance (depth) information can be acquired for each pixel of the image sensor that detects the reflected light, three-dimensional spatial information with relatively high resolution can be constructed.

(How to Use Pattern Light)

The environment map can be generated or updated by capturing an image of pattern light projected from a light source by an imaging unit supported by a part of the arm unit 120 and reconstructing the three-dimensional space on the basis of a shape of the pattern light captured in the image. This method can reconstruct three-dimensional spatial information even under a situation where an object with less change in an image is used as an imaging target, for example. Furthermore, the environment map can be realized at lower cost than the case of using the ToF sensor. Furthermore, by introducing control to perform imaging in a state where the pattern light is projected and imaging in a state where the pattern light is not projected in a time division manner, this method can be realized by providing a light source that project the pattern light to the imaging device (endoscope device), for example. Note that, in this case, for example, an image captured in the state where the pattern light is not projected is only required to be presented to the display device as an image for observing the observation target.

(How to Use Special Light)

There is a procedure performed while observing special light such as narrow band light, auto-fluorescence, infrared light, and the like, and an imaging result of the special light can be used for reconstruction of the three-dimensional space. In this case, for example, it is also possible to record additional information of a lesion, blood vessels, lymph, or the like, in addition to reconstruction of the three-dimensional space.

(Method of Using Polarization Image Sensor)

A polarization image sensor is an image sensor that can detect only a part of polarized light of various types of polarized light contained in incoming light. The environment map can be generated or updated by reconstructing the three-dimensional space using an image captured by such a polarization image sensor.

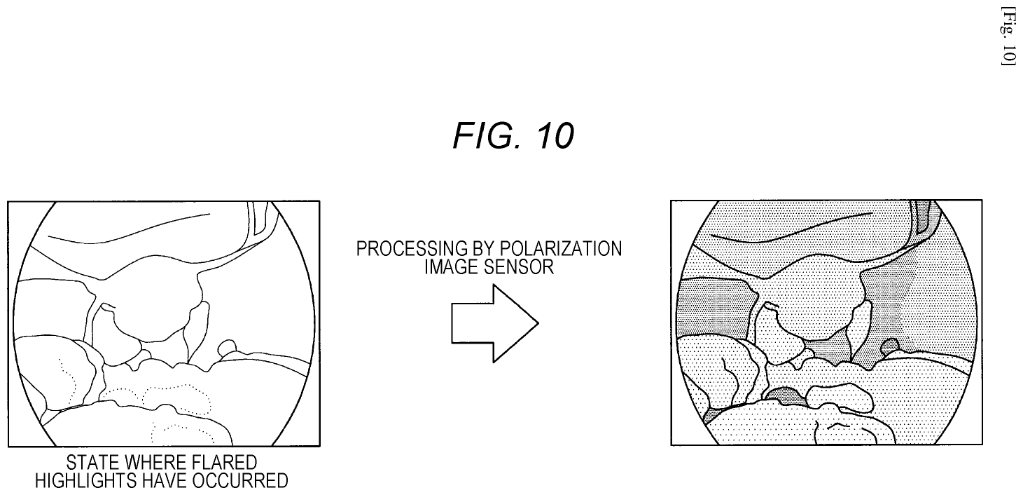

By using this method, a decrease in accuracy regarding the reconstruction of the three-dimensional space due to occurrence of a phenomenon called flared highlight due to a large amount of light can be prevented, for example. Furthermore, as another example, by using the method, the three-dimensional space of an environment where a transparent or translucent object (for example, a body tissue) or an object having a different degree of polarization that is difficult to recognize with naked eyes is present can be more stably reconstructed. For example, FIG. 10 is an explanatory diagram for describing an example of an effect obtained by using the polarization image sensor, illustrating an example of an image captured by the polarization image sensor under a situation where flared highlights occur. The left diagram in FIG. 10 illustrates an example of a case where an image of an observation target is captured using a general image sensor under a situation where the amount of light is relatively large. In other words, in this diagram, flared highlights have occurred. In contrast, the right diagram in FIG. 10 illustrates an example of a case where an image of the observation target is captured using the polarization image sensor under a situation where the amount of light is relatively large, similarly to the left diagram. As can be seen by referring to this diagram, the amount of light to be detected is reduced as compared to the left diagram, and the observation target is more clearly captured. As a result, the accuracy in extracting the characteristic amount of the observation target from the captured image is improved, and the accuracy in reconstructing the three-dimensional space using the captured image can be further improved, accordingly.

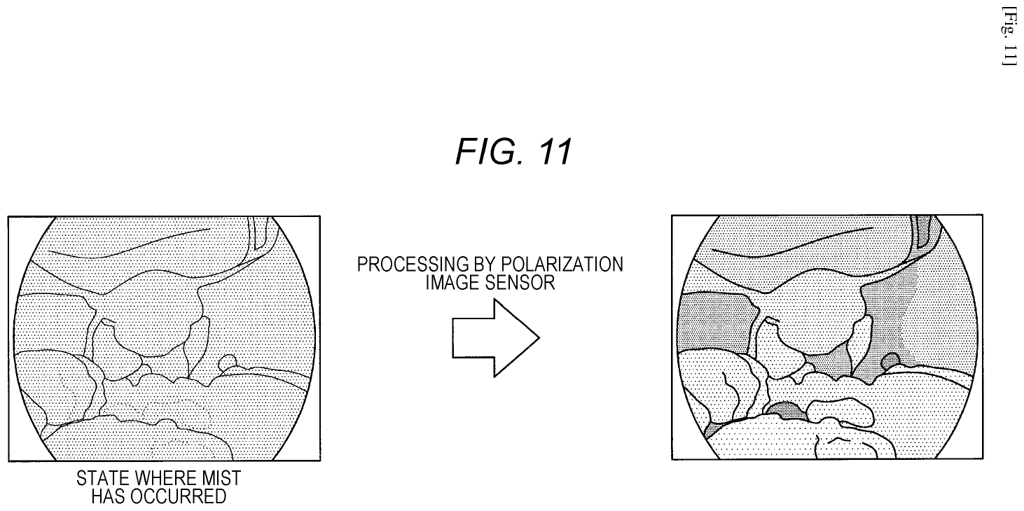

Furthermore, by using this method, for example, even under a situation where noise appears in the captured image or the contrast of the captured image decreases due to occurrence of mist with use of an electric knife or the like, the influence of the mist can be reduced. For example, FIG. 11 is an explanatory diagram for describing an example of an effect obtained by using the polarization image sensor, illustrating an example of an image captured by the polarization image sensor under an environment where the mist has occurred. The left diagram in FIG. 11 illustrates an example of a case where an image of an observation target is captured using a general image sensor under the environment where the mist has occurred. In other words, in the diagram, the contrast is decreased due to the influence of the mist. In contrast, the right diagram in FIG. 11 illustrates an example of a case where an image of the observation target is captured using the polarization image sensor under the environment where the mist has occurred, similarly to the left diagram. As can be seen by referring to this diagram, the decrease in the contrast is suppressed, and the observation target is more clearly captured. As a result, the accuracy in extracting the characteristic amount of the observation target from the captured image is improved, and the accuracy in reconstructing the three-dimensional space using the captured image can be further improved, accordingly.

(Supplement)

Among the above-described methods regarding generation or update of the environment map, two or more methods may be used in combination. As a specific example, a combination of “the method using the captured image” with any of “the method using the distance measurement sensor”, “the method using the pattern light”, “the method using the special light”, and “the method using the polarization image sensor” may be used. In this case, for example, by use of the endoscope device for acquiring the captured image, the above-described combination of methods can be realized by separately providing an acquisition unit (sensor or the like) according to the methods to be applied, in addition to the endoscope device. As described above, by combining a plurality of methods, the accuracy of generation or update of the environment map can be further improved, for example.

Furthermore, not only the above-described information but also other information may be used as long as the other information can be used for estimation of the position and posture of the point of action (in other words, estimation of the self-position) or recognition of the surrounding space. As a specific example, information of an acceleration sensor or an angular velocity sensor that detects change in the position or posture of the point of action (for example, the endoscope) may be used for the estimation of the self-position of the point of action.

Furthermore, the method of acquiring the arm information used for the generation or update of the environment map is also not particularly limited. As a specific example, the arm information according to a recognition result may be acquired by recognizing the state of the arm unit on the basis of an image obtained by capturing the arm unit with an external camera. As a specific example, a marker is attached to each part of the arm unit, and an image obtained by capturing the arm unit with an external camera may be used for recognition of the position and posture of the arm unit (recognition of the position and posture of the point of action, as a result). In this case, it is sufficient that the marker attached to each part of the arm unit is extracted from the captured image, and the position and posture of the arm unit are recognized on the basis of a relationship between the positions and postures of a plurality of the extracted markers.

The example of a method regarding generation or update of the environment map regarding the external environment of the arm unit 120 has been described.

### 5.3. Processing

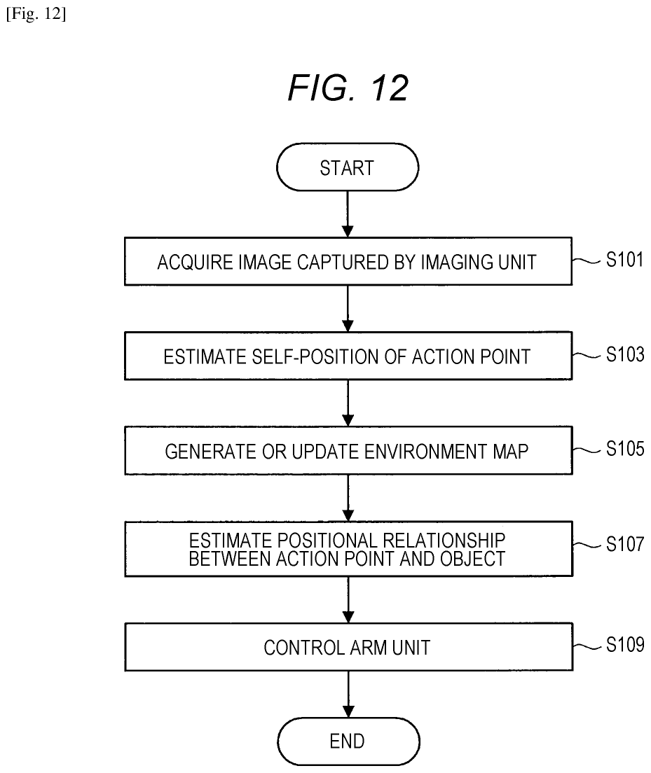

Next, an example of a flow of a series of processing of the control device 20 according to the present embodiment will be described in particular focusing on operations regarding the generation or update of the environment map and the use of the environment map with reference to FIG. 12. FIG. 12 is a flowchart illustrating an example of a flow of a series of processing of the control device 20 according to the present embodiment. Note that, in the present section, an example of a case where the distal end of the endoscope device (imaging unit 140) is set as the point of action, and the generation or update of the environment map is performed using an image captured by the endoscope device will be described.

The control device 20 (operation condition setting unit 242) acquires an image (in other words, the information regarding the space around the endoscope device) captured by the endoscope device (imaging unit 140). The control device 20 extracts the characteristic points from the acquired captured image. As described above, the control device 20 sequentially acquires a captured image by the endoscope device according to the position and posture of the endoscope device (in other words, the point of action), and extracts the characteristic points from the captured image (S101).

The control device 20 (arm state unit 241) acquires, from the support arm device 10, the state (in other words, the arm state) of the arm unit 120 on the basis of the state of the joint unit 130 detected by the joint state detection unit 132. The control device 20 estimates the position and posture of the point of action (for example, the imaging unit 140) in the three-dimensional space (in other words, the self-position of the point of action) on the basis of the acquired arm state (S103).

The control device 20 (operation condition setting unit 242) reconstructs the three-dimensional space on the basis of the correspondence among the characteristic points extracted among the plurality of captured images, and the self-position of the endoscope device (in other words, the self-position of the point of action) at the timing when each of the plurality of captured images is captured. The control device 20 generates the environment map regarding the space around the point of action on the basis of the result of the reconstruction of the three-dimensional space. Furthermore, in a case where the environment map has already been generated at this time, the control device 20 may update the environment map on the basis of the result of the reconstruction of the three-dimensional space. Specifically, the control device 20 may complement a portion where the three-dimensional space has not been generated in the environment map, using the newly reconstructed three-dimensional space information (S105).

Furthermore, the control device 20 (operation condition setting unit 242) estimates the positional relationship between the point of action and an object located around the point of action (for example, a portion such as an organ) on the basis of the generated or updated environment map and the estimation result of the self-position of the point of action (S107). Then, the control device 20 (the virtual force calculation unit 243, the real force calculation unit 244, the ideal joint control unit 250, and the like) controls the operation of the arm unit 120 according to the estimation result of the positional relationship between the point of action and the object (S109).

By applying the above control, the arm control described with reference to FIGS. 7 and 8 (in other words, the arm control in the case of performing an observation using an oblique endoscope) can be realized in a more favorable manner, for example. In other words, in this case, it is sufficient that the operation of the arm unit 120 be controlled such that the state where the observation target is located on the optical axis of the oblique endoscope is maintained according to the relationship of the position and posture between the observation target and the oblique endoscope on the basis of the environment map. Note that an example of another method of controlling the arm unit using the environment map will be separately described below as an example.

An example of the flow of a series of processing of the control device 20 according to the present embodiment has been described in particular focusing on the operations regarding the generation or update of the environment map and the use of the environment map with reference to FIG. 12.

### 5.4. Modification

Next, modifications of the medical arm system 1 according to the present embodiment will be described.

(Modification 1: Configuration Example of Endoscope Device)

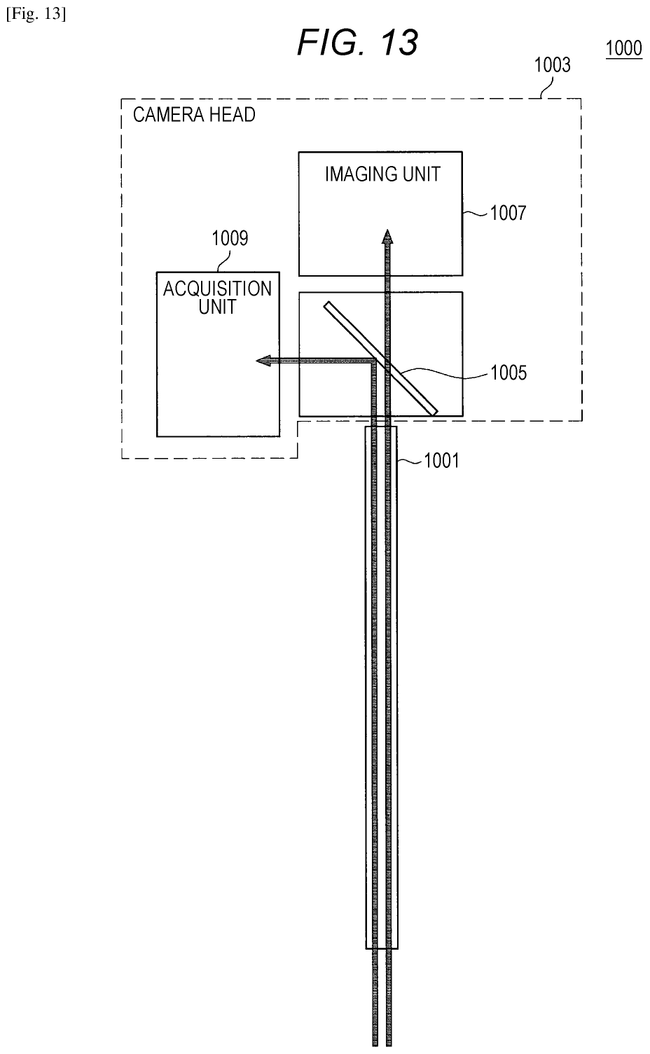

First, as a first modification, an outline of an example of a configuration of an endoscope device supported as the distal end unit by the arm unit 120 in the medical arm system 1 according to the present embodiment will be described. For example, FIG. 13 is an explanatory diagram for describing an example of a schematic configuration of an endoscope device according to the first modification.

In part of the methods of sensing the external environment of the arm unit 120 described as the methods regarding generation or update of the environment map (in particular, in the methods other than the method using the captured image), there are cases where a sensor needs to be separately provided from the endoscope device. Meanwhile, there are cases there installation of a port for inserting the sensor separately from a port for inserting the endoscope device into a body cavity of a patient is difficult from the viewpoint of invasiveness. In such a case, it may be favorable for the endoscope device to acquire the information used for the reconstruction of the three-dimensional space. FIG. 13 discloses a configuration example of an endoscope device for solving such a problem.

Specifically, an endoscope device 1000 illustrated in FIG. 13 includes an endoscope unit 1001 and a camera head 1003. The endoscope unit 1001 schematically illustrates a portion corresponding to a so-called endoscope barrel (in other words, a barrel inserted into the body cavity of the patient). In other words, an image of an observation target (for example, an affected part) acquired by the endoscope unit 1001 is imaged by the camera head 1003.

Furthermore, the camera head 1003 includes a branching optical system 1005, an imaging unit 1007, and an acquisition unit 1009.

The imaging unit 1007 corresponds to a so-called image sensor. In other words, light entering the camera head 1003 via the endoscope unit 1001 forms an image on the imaging unit 1007, so that the image of the observation target is imaged.

The acquisition unit 1009 schematically illustrates a configuration for acquiring the information used for the reconstruction of the three-dimensional space. As a specific example, the acquisition unit 1009 can be configured as the imaging unit (image sensor) or the polarization image sensor described in “5.2. Environment Map Generation Method”.

The branching optical system 1005 can be configured as, for example, a half mirror. In this case, the branching optical system 1005 reflects a part of the light having entered the camera head 1003 via the endoscope unit 1001 and transmits the other part of the light. In other words, the branching optical system partitions a light beam incident onto the branching optical system into a plurality of light beams. In the example illustrated in FIG. 13, the light beam transmitted through the branching optical system 1005 reaches the imaging unit 1007. Thereby, the image of the observation target is captured. Furthermore, the light beam reflected by the branching optical system 1005 reaches the acquisition unit 1009. The three-dimensional space is reconstructed on the basis of the information acquired by the acquisition unit 1009, and the environment map is generated or updated using the result of the reconstruction, under such a configuration.

Furthermore, the branching optical system 1005 may be configured as a color separation optical system configured using an optical film that separates incident light according to wavelength characteristics such as a dichroic film. In this case, the branching optical system 1005 reflects light belonging to a part of a wavelength band and transmits light belonging to the other part of the wavelength band, among the light having entered the camera head 1003 through the endoscope unit 1001. With such a configuration, for example, among the light having entered the camera head 1003, light belonging to a visible light region can be guided to the imaging unit 1007 and light belonging to another wavelength band (for example, infrared light or the like) can be guided to the acquisition unit 1009.

Note that at least one of the imaging unit 1007 or the acquisition unit 1009 may be configured to be detachable from the camera head 1003. With such a configuration, for example, a device to be applied as at least one of the imaging unit 1007 or the acquisition unit 1009 can be selectively switched according to a procedure to be performed or a method of observing the observation target.

As the first modification, an outline of an example of the configuration of the endoscope device supported as the distal end unit by the arm unit 120 in the medical arm system 1 according to the present embodiment has been described with reference to FIG. 13.

(Modification 2: Control Example Regarding Acquisition of Information Using Imaging Unit)

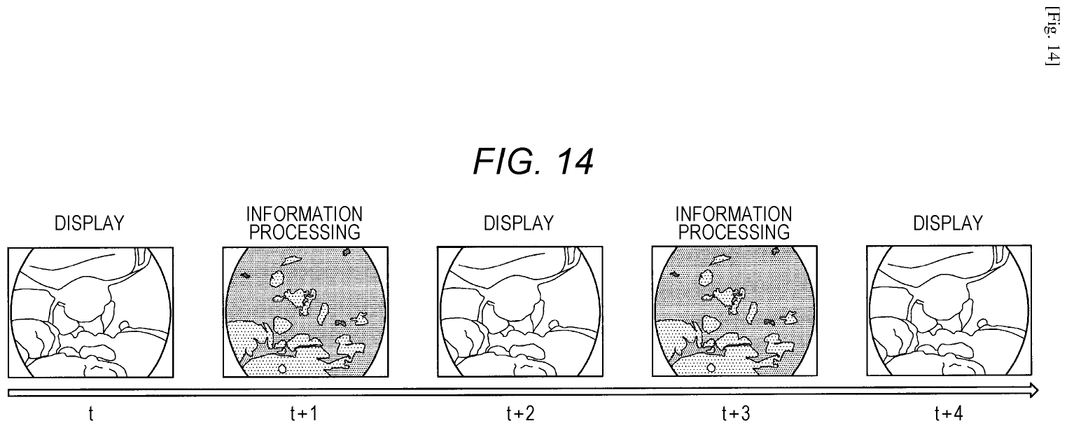

Next, as a second modification, an example of a control method for individually acquiring both an image to be used for the observation of the observation target and an image to be used for the generation or update of the environment map, using an imaging unit such as an endoscope device, will be described. For example, FIG. 14 is an explanatory diagram for describing an outline of an operation of a medical arm system according to the second modification, illustrating an example of control regarding acquisition of information used for the generation or update of the environment map.

In the example illustrated in FIG. 14, the endoscope device (imaging unit) acquires an image to be used for the observation of the observation target (in other words, an image to be presented via an output unit such as a display) and an image to be used for the generation or update of the environment map in a time division manner. Specifically, images acquired at timings t, t+2, and t+4 are presented to the surgeon (user) by being displayed on the display unit. In contrast, images acquired at timings t+1 and t+3 are used for processing regarding the generation or update of the environment map. In other words, the imaging unit captures an image of the space surrounding the point of action at specified time intervals and each of these images are used for processing regarding the generation or update of the environment map. In other words, extraction of characteristic points from the images, the reconstruction of the three-dimensional space based on the extraction result of the characteristic points, and the generation or update of the environment map using the reconstruction of the three-dimensional space are performed.

By applying the above control, both the display of the imaging result of the observation target and the generation or update of the environment map can be realized without separately providing a sensor to the endoscope device.

As the second modification, the example of a control method for individually acquiring both an image to be used for the observation of the observation target and an image to be used for the generation or update of the environment map, using an imaging unit such as an endoscope device, has been described with reference to FIG. 14.

(Modification 3: Application Example of Mask Processing)

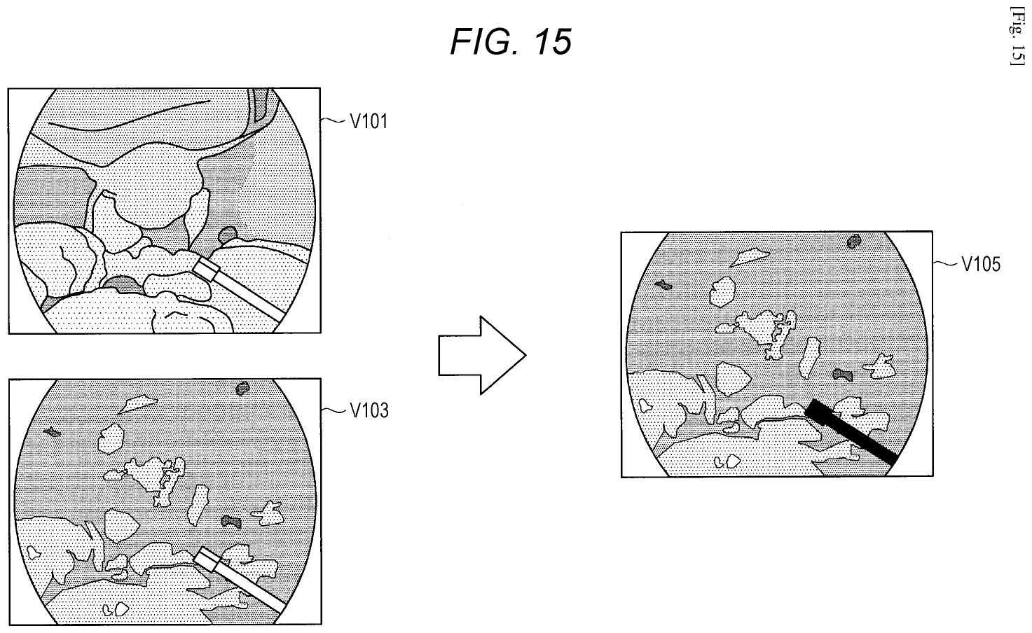

Next, as a third modification, an example of processing of excluding a part of acquired information of a surrounding environment from a target for the reconstruction of the three-dimensional space (in other words, a target for the generation or update of the environment map) will be described. For example, FIG. 15 is an explanatory diagram for describing an outline of an operation of a medical arm system according to the third modification, illustrating an example of control regarding acquisition of information used for the generation or update of the environment map.

FIG. 15 illustrates an image V101 captured by the endoscope device (imaging unit). In other words, the example in FIG. 15 illustrates a situation in which various types of treatment are performed for an affected part while observing a body cavity of a patient using the image V101 captured by the endoscope device. Under such a situation, there are some cases where another object such as a medical instrument used for applying treatment to the affected part, other than a site (for example, an organ or the like) in the body cavity of the patient, is captured in the image, in addition to the portion in the body cavity of the patient. For example, a medical instrument is captured in addition to the site in the body cavity of the patient in the image V101. Under such a situation, there are some cases where information regarding the medical instrument is acquired for the information to be used for the reconstruction of the three-dimensional space around the point of action (in other words, the information to be used for the generation or update of the environment map). For example, information V103 is information used for the reconstruction of the three-dimensional space. In other words, in the example illustrated in FIG. 15, information regarding a medical instruments (for example, an extraction result of characteristic points of the medical instrument) is acquired in addition to the information regarding the site in the body cavity of the patient (for example, an extraction result of the characteristic points of the portion) in the information V103.

Meanwhile, with regard to the medical instrument, due to the characteristic that the position and posture are changed by an operation of the surgeon, the frequency of change in the position and posture changing is higher than the frequency of the site in the body cavity of the patient. If such a frequently moving object is targeted for the generation or update of the environment map, it can be assumed that a processing load associated with the generation or update of the environment map increases, and affects other processing, accordingly. In view of such a situation, an object having a large frequency in change in the position and posture may be excluded from the target for the reconstruction of the three-dimensional space (in other words, the target for the generation or update of the environment map). Furthermore, not only the medical instrument but also objects (solids, liquids, or the like) having a high frequency in change in the position, posture, shape, or the like, such as blood, may be excluded from the target for the reconstruction of the three-dimensional space.

Note that the excluding method is not particularly limited as long as the information regarding the objects to be excluded (for example, the medical instrument, blood, and the like) can be specified from the information to be used for the reconstruction of the three-dimensional space around the point of action. As a specific example, the position and posture of the medical instrument can be recognized on the basis of the arm information according to the state (for example, the position and posture) of the arm unit 120 supporting the medical instrument. As a specific example, the position and posture of the medical instrument in the captured image can be recognized according to a relative relationship between an imaging range of the endoscope device recognized on the basis of the position and posture of the endoscope device and the position and posture of the medical instrument. Furthermore, the position and posture of the object to be excluded can be recognized by detecting a shape characteristic or a color characteristic of the object. Mask processing may be applied to a region corresponding to the object to be excluded by specifying the region corresponding to the object in the information to be used for the reconstruction of the three-dimensional space around the point of action from the recognition result of the position and posture of the object, which has been obtained as described above. Furthermore, as another example, information with a change amount in the position and posture exceeding a threshold value (for example, a characteristic point with a moving amount exceeding a threshold value), of the information to be used for the reconstruction of the three-dimensional space around the point of action, may be excluded from the target for the reconstruction of the three-dimensional space.

As the third modification, the example of processing of excluding a part of acquired information of a surrounding environment from a target of the reconstruction of the three-dimensional space (in other words, a target of the generation or update of the environment map) has been described with reference to FIG. 15.

### 5.5. Example

Next, examples of the operation of the medical arm system 1 according to the present embodiment will be described by taking specific examples.

### First Example: Force Control Using Environment Map

First, as a first example, an example of recognizing a positional relationship between an observation target and a point of action using an environment map, and performing force control of an arm unit according to a recognition result of the positional relationship will be described.

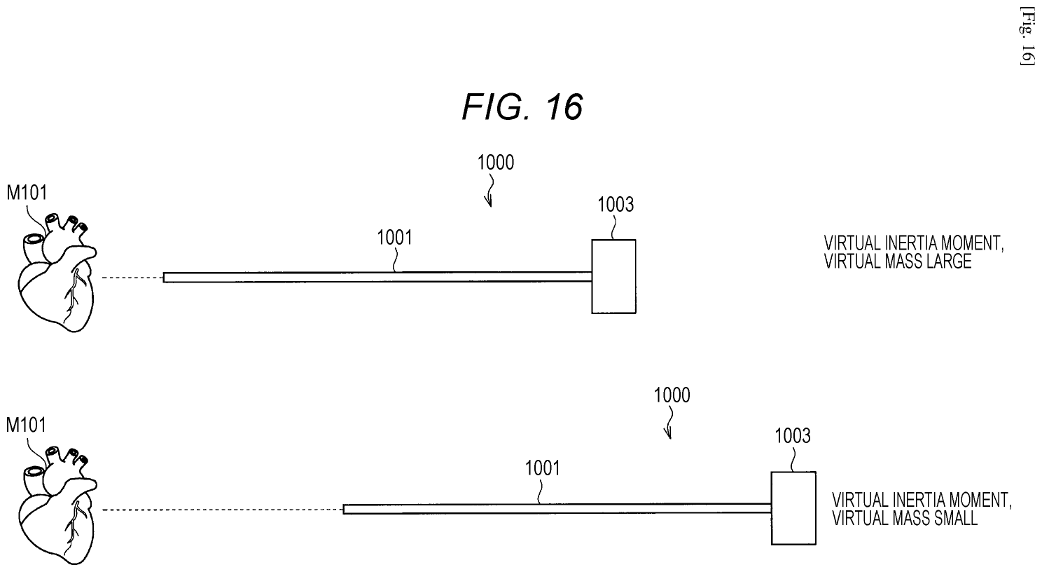

For example, FIG. 16 is an explanatory diagram for describing an overview of an example of arm control according to the first example. FIG. 16 illustrates the endoscope device 1000. In other words, the endoscope unit 1001 and the camera head 1003 of the endoscope device 1000 are illustrated. Furthermore, a site (for example, an organ or the like) M101 in a body cavity of a patient is schematically illustrated.

In the arm control according to the first example, parameters regarding force control of the arm unit 120 that supports the endoscope device 1000 are adjusted according to the positional relationship between the site M101 to be observed and the distal end (in other words, the point of action) of the endoscope unit 1001.

Specifically, as illustrated in the upper drawing in FIG. 16, virtual inertia moment and a virtual mass regarding in the control of the arm unit 120 may be controlled to be larger in a case where the distance between the site M101 and the distal end of the endoscope unit 1001 is short (for example, the distance is equal to or smaller than a threshold value). In other words, in this case, the parameters are adjusted such that the surgeon who operates the endoscope device 1000 feels that the inertia and mass of the endoscope are heavier than in reality, thereby reducing influence of camera shake at the time of direct operation.

In contrast, as illustrated in the lower drawing in FIG. 16, the virtual inertia moment and the virtual mass regarding in the control of the arm unit 120 may be controlled to be smaller in a case where the distance between the site M101 and the distal end of the endoscope unit 1001 is large (for example, the distance exceeds the threshold value). In other words, in this case, the parameters are adjusted such that the surgeon who operates the endoscope device 1000 feels that the inertia and mass of the endoscope are lighter than in reality, thereby realizing a light operation feeling and reducing an operation load.

Furthermore, the operation of the arm unit 120 may be controlled to make friction parameters such as coulomb friction and viscous friction larger in the case where the distance between the site M101 and the tip of the endoscope unit 1001 is short. With the control, even in a case where a strong force is unexpectedly applied to the endoscope device 1000, a rapid change in the position and posture can be suppressed. Furthermore, the operation of the arm unit 120 can be controlled such that a state where a fixed force is being applied to the endoscope device 1000 is maintained without causing the surgeon (operator) to adjust a delicate force under a situation where the endoscope device 1000 is moved at a constant speed.

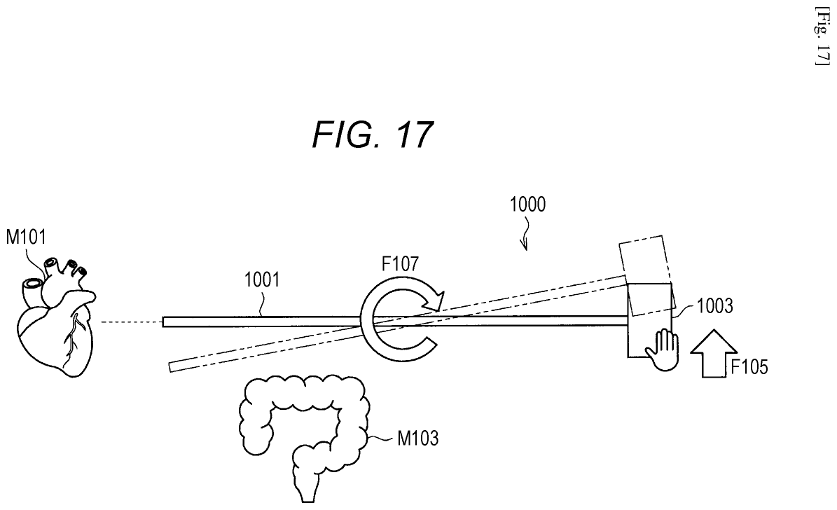

Furthermore, FIG. 17 is an explanatory diagram for describing an overview of another example of the arm control according to the first example. In FIG. 17, similar reference numerals to FIG. 16 similarly represent the objects denoted with the same reference numerals in the example illustrated in FIG. 16. Furthermore, a site (for example, an organ or the like) M103 in a body cavity of a patient is schematically illustrated, and corresponds to another site different from the site M101.

For example, the example in FIG. 17 schematically illustrates a situation in which the surgeon has a difficulty in confirming the presence of the site M103 from the image captured by the endoscope device 1000. Even in such a situation, the positional relationship between the site M103 and the endoscope device 1000 is recognized on the basis of the environment map, for example, so that the above-described kinetic parameters can be adjusted to avoid the contact between the site M103 and the endoscope device 1000. As a specific example, as illustrated in FIG. 17, in a case where the surgeon operates the endoscope device 1000, the operation of the arm unit 120 is controlled to generate a reaction force F107 to cancel a force F105 added to the endoscope device 1000 by the operation, so that the contact between the endoscope device 1000 and the site M103 can be avoided.

As the first example, the example of recognizing a positional relationship between an observation target and a point of action using an environment map, and performing force control of an arm unit according to a recognition result of the positional relationship has been described with reference to FIGS. 16 and 17.

### Second Example: Speed Control Using Environment Map

Next, as a second example, an example of recognizing a positional relationship between an observation target and a point of action using an environment map, and performing speed control of a point of action according to a recognition result of the positional relationship will be described.

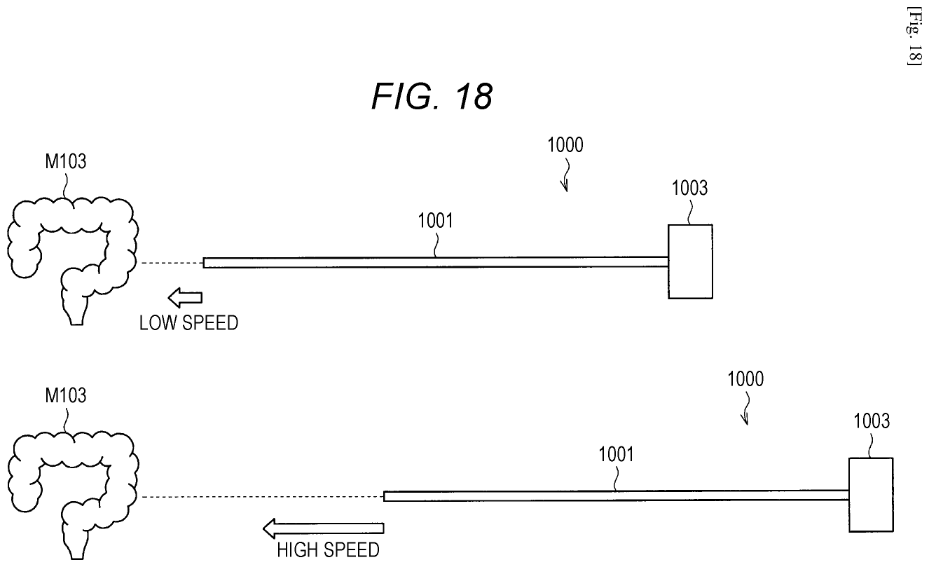

For example, FIG. 18 is an explanatory diagram for describing an overview of an example of arm control according to the second example. In FIG. 18, similar reference numerals to FIGS. 16 and 17 similarly represent the objects denoted with the same reference numerals in the example illustrated in FIGS. 16 and 17.

In the arm control according to the second example, an insertion speed of the endoscope device 1000 is controlled according to the positional relationship between the site M103 to be observed and the distal end (in other words, the point of action) of the endoscope unit 1001 under a situation where insertion of the endoscope device 1000 is performed by remote control, an audio instruction, or the like.

Specifically, as illustrated in the upper drawing in FIG. 18, the insertion speed of the endoscope device 1000 may be controlled to be slower (for example, the insertion speed becomes equal to or smaller than a threshold value) in the case where the distance between the site M103 and the distal end of the endoscope unit 1001 is short (for example, the distance is equal to or smaller than a threshold value). In contrast, as illustrated in the lower drawing in FIG. 18, the insertion speed of the endoscope device 1000 may be controlled to be faster (for example, the insertion speed exceeds the threshold value) in the case where the distance between the site M103 and the distal end of the endoscope unit 1001 is long (for example, the distance exceeds the threshold value).

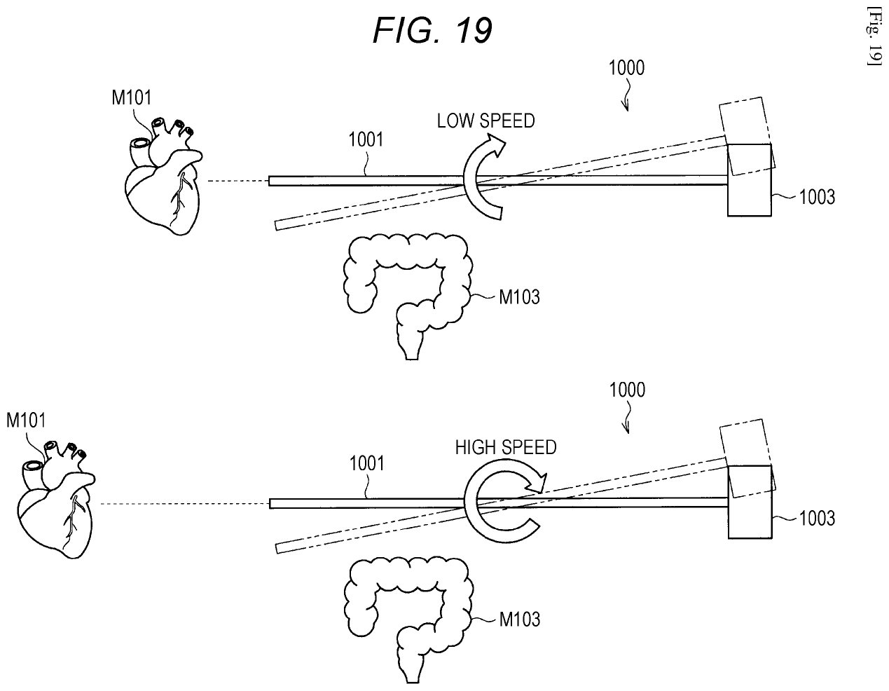

Furthermore, FIG. 19 is an explanatory diagram for describing an overview of another example of the arm control according to the second example. In FIG. 19, similar reference numerals to FIGS. 16 and 17 similarly represent the objects denoted with the same reference numerals in the example illustrated in FIGS. 16 and 17.

For example, the example in FIG. 19 schematically illustrates a situation in which the surgeon has a difficulty in confirming the presence of the site M103 from the image captured by the endoscope device 1000. Even in such a situation, the positional relationship between the site M103 and the endoscope device 1000 is recognized on the basis of the environment map, for example, so that the speed regarding the change in the position and posture of the endoscope device 1000 may be controlled for the purpose of avoiding the contact between the site M103 and the endoscope device 1000.

As a specific example, as illustrated in the upper drawing in FIG. 19, a speed regarding the position and posture of the endoscope device 1000 may be controlled to be slower (for example, the speed becomes equal to or smaller than a threshold value) in the case where the distance between each of the site M101 and M103 and the distal end of the endoscope unit 1001 is short (for example, the distance is equal to or smaller than the threshold value). In contrast, as illustrated in the lower drawing in FIG. 19, the speed regarding the position and posture of the endoscope device 1000 may be controlled to be faster (for example, the speed exceeds the threshold value) in the case where the distance between each of the site M101 and M103 and the distal end of the endoscope unit 1001 is long (for example, the distance exceeds the threshold value).

As the second example, the example of recognizing a positional relationship between an observation target and a point of action using an environment map, and performing speed control of a point of action according to a recognition result of the positional relationship has been described with reference to FIGS. 18 and 19.

### Third Example: Adjustment of Control Amount Using Environment Map

Next, as a third example, an example of recognizing a positional relationship between an observation target and a point of action using an environment map, and adjusting a control amount regarding change in position and posture of an arm unit according to a recognition result of the positional relationship will be described.

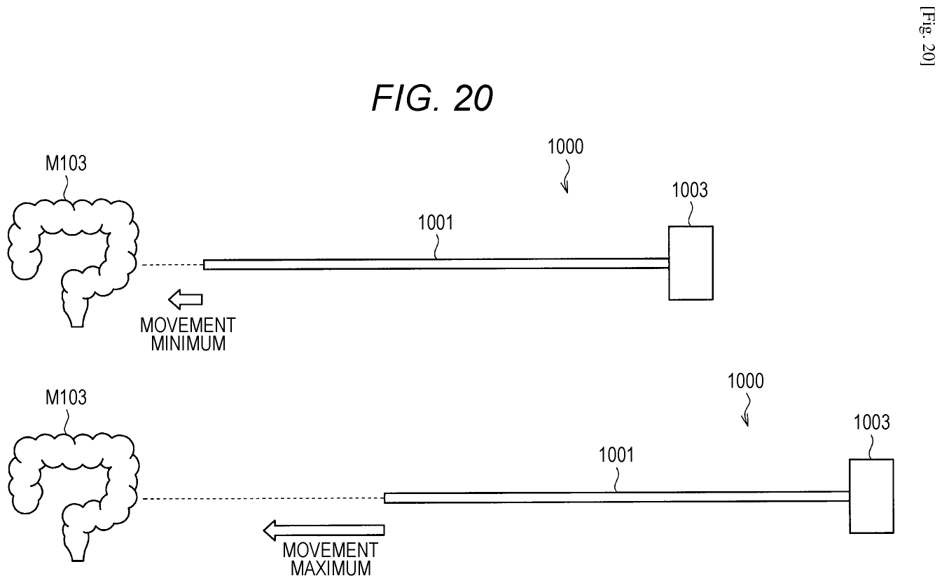

For example, FIG. 20 is an explanatory diagram for describing an overview of an example of arm control according to the third example. In FIG. 20, similar reference numerals to FIGS. 16 and 17 similarly represent the objects denoted with the same reference numerals in the example illustrated in FIGS. 16 and 17.

In the arm control according to the third example, a moving amount regarding insertion of the endoscope device 1000 is controlled according to the positional relationship between the site M103 to be observed and the distal end (in other words, the point of action) of the endoscope unit 1001 under a situation where the insertion of the endoscope device 1000 is performed by remote control, an audio instruction, or the like.

Specifically, as illustrated in the upper drawing in FIG. 20, the moving amount regarding the insertion of the endoscope device 1000 may be adjusted to be smaller (for example, the moving amount becomes equal to or smaller than a threshold value) in the case where the distance between the site M103 and the distal end of the endoscope unit 1001 is short (for example, the distance is equal to or smaller than a threshold value). In contrast, as illustrated in the lower drawing in FIG. 20, the moving amount regarding the insertion of the endoscope device 1000 may be adjusted to be larger (for example, the moving amount exceeds the threshold value) in the case where the distance between the site M103 and the distal end of the endoscope unit 1001 is long (for example, the distance exceeds the threshold value).

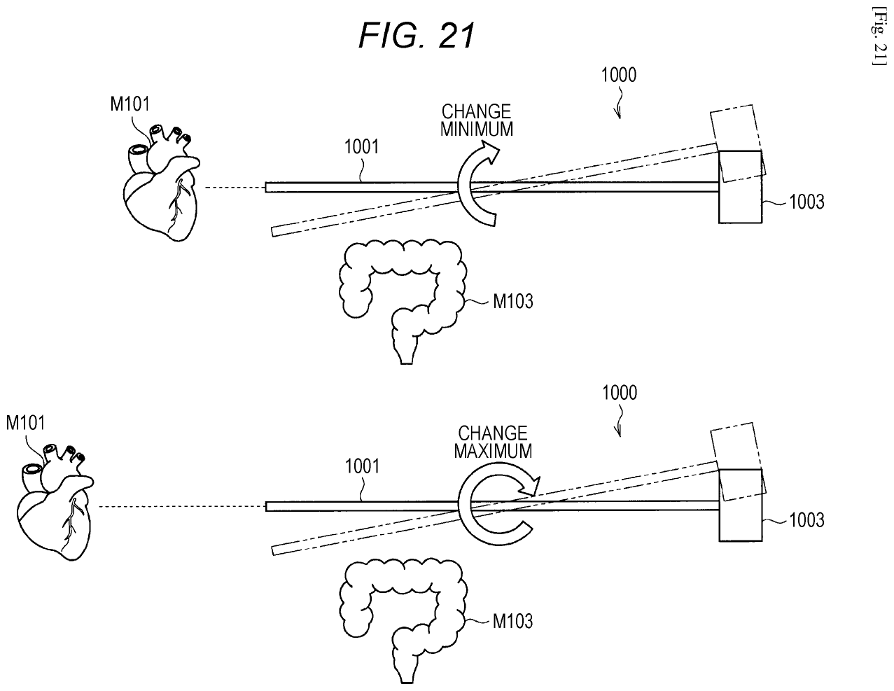

Furthermore, FIG. 21 is an explanatory diagram for describing an overview of another example of the arm control according to the third example. In FIG. 21, similar reference numerals to FIGS. 16 and 17 similarly represent the objects denoted with the same reference numerals in the example illustrated in FIGS. 16 and 17.

For example, the example in FIG. 21 schematically illustrates a situation in which the surgeon has a difficulty in confirming the presence of the site M103 from the image captured by the endoscope device 1000. Even in such a situation, the positional relationship between the site M103 and the endoscope device 1000 is recognized on the basis of the environment map, for example, so that the control amount regarding the change in the position and posture of the endoscope device 1000 may be controlled for the purpose of avoiding the contact between the site M103 and the endoscope device 1000.

As a specific example, as illustrated in the upper drawing in FIG. 21 a control amount (change amount) regarding change in the position and posture of the endoscope device 1000 may be adjusted to be smaller (for example, the control amount becomes equal to or smaller than a threshold value) in the case where the distance between each of the site M101 and M103 and the distal end of the endoscope unit 1001 is short (for example, the distance is equal to or smaller than the threshold value). In contrast, as illustrated in the lower drawing in FIG. 21, the control amount (change amount) regarding change in the position and posture of the endoscope device 1000 may be adjusted to be larger (for example, the control amount exceeds the threshold value) in the case where the distance between each of the site M101 and M103 and the distal end of the endoscope unit 1001 is long (for example, the distance exceeds the threshold value).

As the third example, the example of recognizing a positional relationship between an observation target and a point of action using an environment map, and adjusting a control amount regarding change in position and posture of an arm unit according to a recognition result of the positional relationship has been described with reference to FIGS. 20 and 21.

### Fourth Example: Control Example of Moving Route Using Environment Map

Next, as a fourth example, an example of a case of planning a route to move a point of action toward an observation target and controlling the route at the time of moving the point of action using an environment map will be described.

The position and posture of a site difficult to recognize from the image captured by the endoscope device 1000 can be recognized using an environment map generated in advance. By using such a characteristic, the route of the movement can be planned in advance in moving the endoscope device 1000 to a position where a desired site (observation target) is observable.

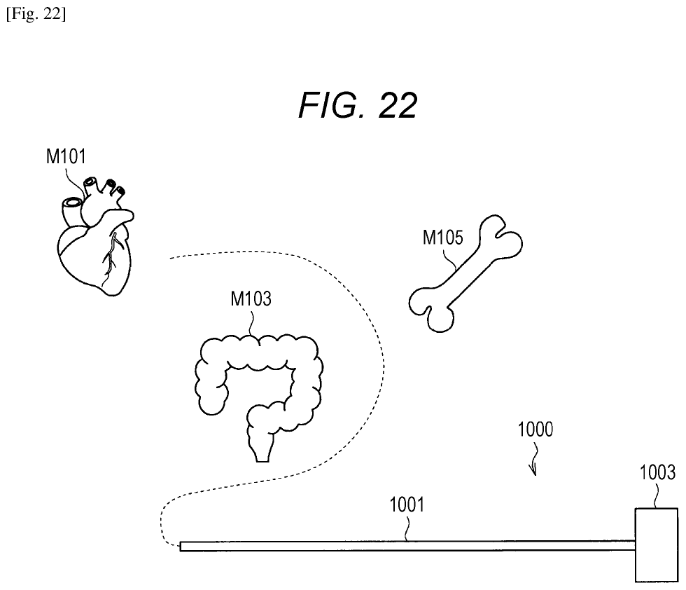

For example, FIG. 22 is an explanatory diagram for describing an overview of another example of arm control according to a fourth example. In FIG. 22, similar reference numerals to FIGS. 16 and 17 similarly represent the objects denoted with the same reference numerals in the example illustrated in FIGS. 16 and 17. Furthermore, a site (for example, an organ or the like) M105 in a body cavity of a patient is schematically illustrated, and corresponds to another site different from the sites M101 and M103.

FIG. 22 schematically illustrates a situation in which the endoscope device 1000 is moved to a position where the site M101 is observable, using the site M101 as the observation target. Furthermore, in the example illustrated in FIG. 22, sites M103 and M105 are present in addition to the site M101 to be observed. Even under such a situation, the respective positions and postures of the sites M101, M103, and M105 can be recognized in advance by using the environment map generated in advance.

Therefore, the route to move the endoscope device 1000 to the position where the site M101 is observable can be planned in advance while avoiding a contact between each of the sites M103 and M105 with the endoscope device 1000, by using the recognition result. Furthermore, even under the situation where the endoscope device 1000 is moved to the position where the site M101 is observable, the endoscope device 1000 can be controlled to be moved along the route.

As the fourth example, the example of a case of planning a route to move a point of action toward an observation target and controlling the route at the time of moving the point of action using an environment map has been described with reference to FIG. 22.

### Fifth Example: Acceleration Control Using Environment Map

Next, as a fifth example, an example of recognizing a positional relationship between an observation target and a point of action using an environment map, and performing acceleration control of a point of action according to a recognition result of the positional relationship will be described.

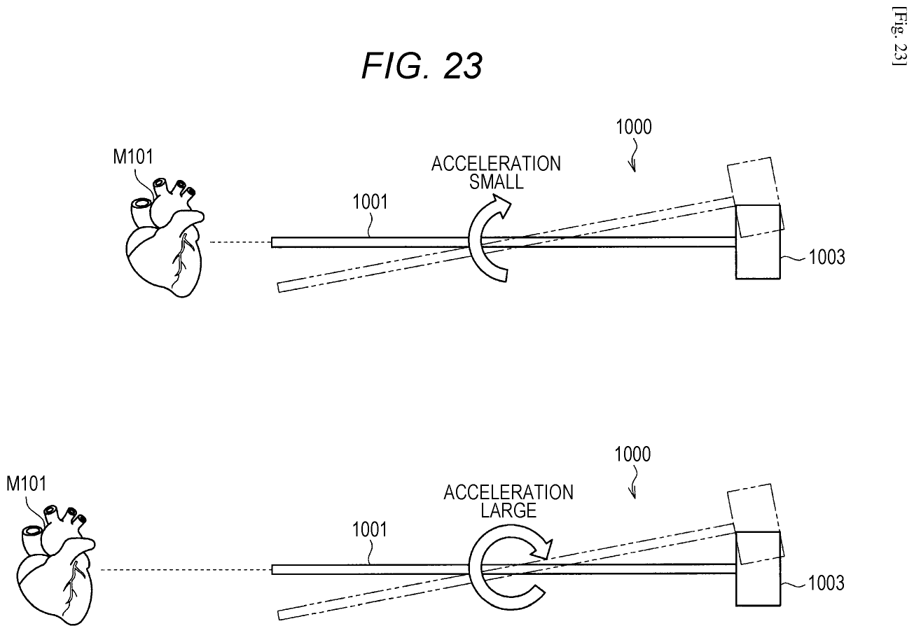

For example, FIG. 23 is an explanatory diagram for describing an overview of an example of arm control according to the fifth example. In FIG. 23, similar reference numerals to FIGS. 16 and 17 similarly represent the objects denoted with the same reference numerals in the example illustrated in FIGS. 16 and 17.

In the arm control according to the fifth example, acceleration regarding change in the position and posture of the endoscope device 1000 is controlled according to the positional relationship between the site M103 to be observed and the distal end (in other words, the point of action) of the endoscope unit 1001 under a situation where insertion of the endoscope device 1000 is performed by remote control, an audio instruction, or the like.

Specifically, as illustrated in the upper drawing in FIG. 23, the acceleration regarding change in the position and posture of the endoscope device 1000 may be controlled to be smaller (for example, the acceleration becomes equal to or smaller than a threshold value) in the case where the distance between the site M101 and the distal end of the endoscope unit 1001 is short (for example, the distance is equal to or smaller than a threshold value). In contrast, as illustrated in the lower drawing in FIG. 23, the acceleration regarding change in the position and posture of the endoscope device 1000 may be controlled to be larger (for example, the acceleration exceeds the threshold value) in the case where the distance between the site M101 and the distal end of the endoscope unit 1001 is long (for example, the distance exceeds the threshold value).

In a case where the operation of the position and posture of the endoscope device 1000 is performed using an operation device such as a remote controller or a joystick by the above control, a feedback for the operation can be changed according to the situation at each time. Thereby, for example, the weight of the operation can be fed back in a pseudo manner to the surgeon (operator).

As the fifth example, the example of recognizing a positional relationship between an observation target and a point of action using an environment map, and performing acceleration control of a point of action according to a recognition result of the positional relationship has been described with reference to FIG. 23.

### Sixth Example: Example of Control According to Surface Shape of Object

Next, as a sixth example, an example of a case of recognizing a surface shape of an observation target using an environment map, and controlling position and posture of a point of action according to a relationship of position and generation between a surface of the observation target and the point of action will be described.

As described above, the position, posture, shape of an object located around the point of action (for example, the endoscope or the like) can be recognized using the generated or updated environment map. In other words, the surface shape of the object can be recognized. The operation of the arm unit can be controlled such that the point of action (for example, a distal end of a medical instrument or the like) moves along the surface of the object, for example, using such a characteristic.

Furthermore, the operation of the arm unit may be controlled such that change in the posture of the point of action with respect to the surface of the object (in other words, a normal vector of the surface) falls within a predetermined range. As a specific example, the posture of the endoscope may be controlled such that change in an angle made by the optical axis of the endoscope and the normal vector of the surface at a point on the surface of the object located on the route of the optical axis falls within a predetermined range. Such control enables suppression of the change in the angle at which the observation target is observed.

Furthermore, as another example, the posture of the endoscope device (for example, a direction in which the optical axis of the endoscope is directed) may be controlled according to the posture of a surgical tool with respect to the surface of the object to be observed (in other words, the normal vector of the surface). Such control enables control of the posture of the endoscope such that a camera angle with respect to the observation target becomes in a favorable state according to the state of the surgical tool.

As the sixth example, the example of a case of recognizing a surface shape of an observation target using an environment map, and controlling position and posture of a point of action according to a relationship of position and generation between a surface of the observation target and the point of action has been described.

### Seventh Example: Example of Control According to Reliability of Acquired Information

Next, as a seventh example, an example of evaluating reliability (probability) of information of a surrounding space acquired by an imaging unit (endoscope) or the like and controlling generation or update of an environment map according to an evaluation result will be described.

For example, there are some cases where recognition of an object captured in an image according to an imaging condition is difficult under a situation where the image captured by an imaging unit (endoscope or the like) is used for generation or update of an environment map. As a specific example, in a case where a phenomenon called “flared highlights” in which the image is captured brighter (for example, the luminance exceeds a threshold value) or conversely a phenomenon called “blocked up shadows” in which the image is captured darker (for example, the luminance is equal to or smaller than the threshold value) has occurred, there are some cases where the contrast is decreased or a signal-to-noise ratio (SN ratio) becomes lower. In such a case, a case where recognition or identification of the object in the image becomes difficult is assumed, and the reliability (probability) of characteristic points extracted from the image tends to be lowered as compared with appropriate exposure, for example. In view of such a situation, the reliability of the information may be associated with information used for the generation or update of the environment map.

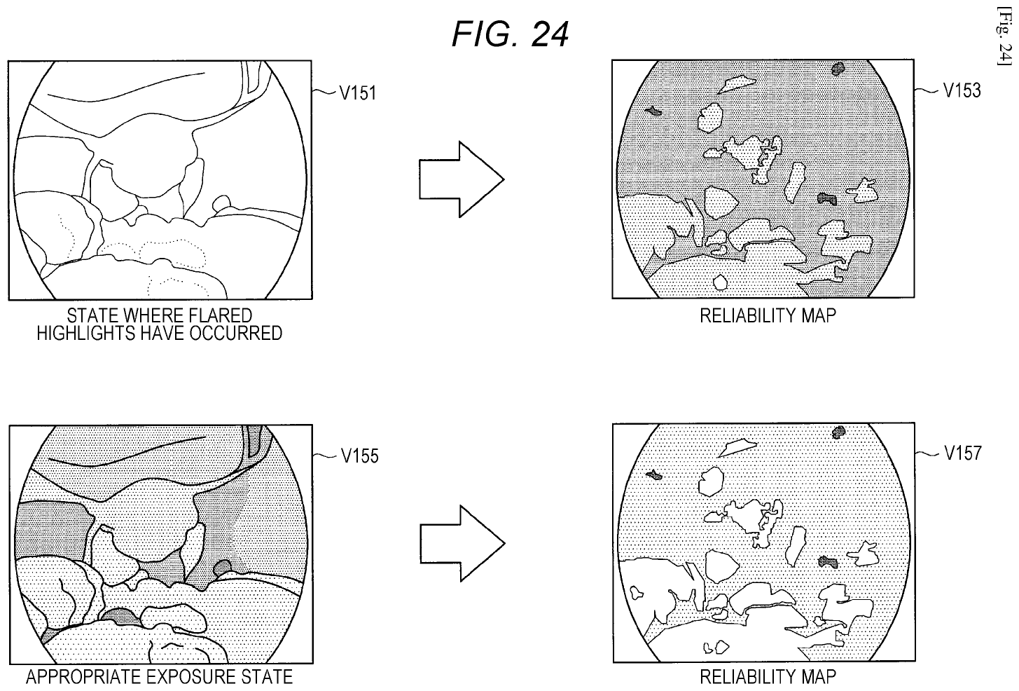

For example, FIG. 24 is an explanatory diagram for describing an example of control regarding the generation or update of the environment map according to the seventh example. The example in FIG. 24 illustrates an example of a reliability map indicating the reliability of the image in a case of using the image captured by the imaging unit (endoscope) for the generation or update of the environment map. In FIG. 24, an image V151 is an image captured in a state where flared highlights have occurred. In contrast, an image V155 is an image captured in appropriate exposure. Furthermore, information V153 and V157 is information (hereinafter also referred to as “reliability map”) obtained by mapping reliability of information corresponding to respective pixels of the images V151 and V155 in a two-dimensional manner. Note that, in the reliability maps V153 and V157, the information of the pixels is set such that the brighter the pixel with higher reliability. In other words, it is found that the reliability map V155 corresponding to the image V151 in which the flared highlights have occurred is darker in brightness of each pixel and is lower in the reliability than the reliability map V157 corresponding to the image V153 captured in appropriate exposure.

An environment map with higher accuracy can be constructed by controlling whether or not using the acquired information regarding a surrounding space for the generation or update of the environment map on the basis of the above reliability. As a specific example, in a case where the reliability of newly acquired information is higher than information (for example, characteristic points) already applied to an environment map, the environment map may be updated on the basis of the acquired information. In contrast, in a case where the reliability of the newly acquired information is lower than the information already applied to the environment map, the update of the environment map may be suppressed based on the acquired information. By updating the environment map by the above control, a more reliable environment map (for example, an environment map with a smaller error from the real space) can be constructed.

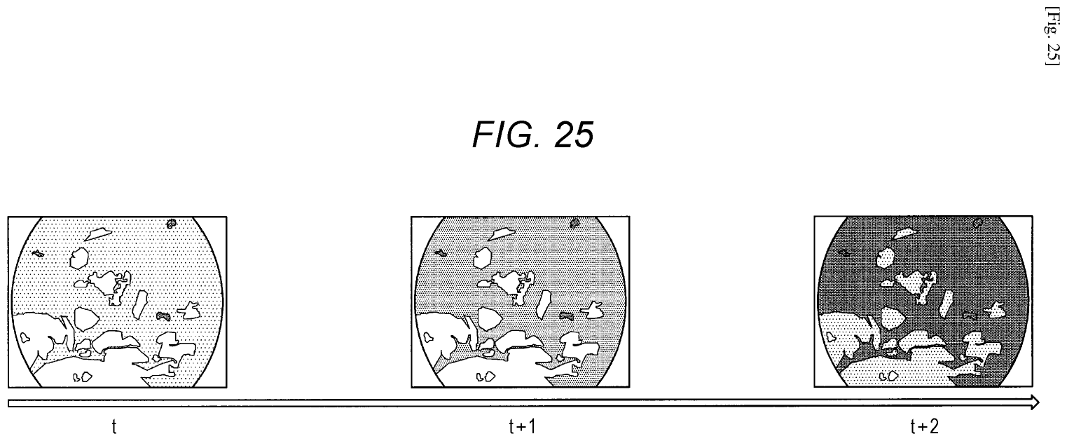

Note that a situation where the surrounding environment changes from hour to hour. Under such a situation, a situation where the reliability of information is further lowered as the time further passes from timing when the information has been acquired can be assumed. Therefore, for example, even in the case of using the acquired information regarding a surrounding space for the generation or update of the environment map, the generation or update of the environment map considering time change in the surrounding space can be realized by decreasing the reliability of the information over time. For example, FIG. 25 is an explanatory diagram for describing an example of control regarding the generation or update of the environment map according to the seventh example, illustrating an example of the reliability map in a case where the control to decrease the reliability map over time is applied.

Note that the control of the reliability considering the temporal change may be performed to uniformly decrease a predetermined value in the entire environment map or may be performed to have a bias decreased according to various conditions. As a specific example, in a case of controlling the reliability of a generated environment map for the environment map of a body cavity of a patient, a value of the reliability decreased according to a tissue or a type of a site may be controlled, for example. More specifically, since bone has less temporal change than an organ or the like, the value of the reliability to be decreased may be set to be smaller in a portion corresponding to the bone in the environment map than in a portion corresponding to the organ. Furthermore, since the temporal change tends to be relatively larger in the vicinity of the site to which treatment is applied in surgery than the other sites, the value of the reliability may be set to be lower in the vicinity of the site than in the other sites.

Furthermore, the environment map may be constructed in advance using a CT image, an MRI image, a human body mode, or the like. In such a case, the reliability associated with the environment map may be set to be sufficiently lower than the reliability of a case where information is acquired by a direct observation with an endoscope or the like. Furthermore, in a case of constructing an environment map of a human body in advance, various types of information regarding the human body may be used for the construction of the environment map. As a specific example, approximate positions of various organs can be estimated using information such as height, weight, chest circumference, and abdominal circumference, so the estimation result may be reflected in the environment map.

Here, an example of a method of using the environment map according to the present embodiment will be described focusing on a case where the operation of the endoscope device supported by the arm unit is performed. For example, in prostate cancer surgery, the site to be treated tends to be extensive, so a situation can be assumed where the endoscope is moved each time according to a location to be treated. Under such a situation, in a case where the reliability of information in the environment map corresponding to a position to which the distal end of the endoscope is to be moved is low, a possibility of presence of a site where information has not been acquired at the time of generation or update of the environment map may be high. Under such a situation, when the endoscope is moved at high speed, there is a possibility that the endoscope comes in contact with the site where information has not been acquired. Therefore, in such a case, the moving speed of the environment map is set to be low, and in a case where the reliability of a portion corresponding to the site in the environment map becomes high due to new acquisition of information, the moving speed of the endoscope may be controlled again (for example, the endoscope may be controlled to be move faster). By the control, the observation can be more safely performed while avoiding a contact between the endoscope and a site in the body.

Furthermore, the information regarding the reliability can also be used for parameter adjustment of force control. As a specific example, at a position with high reliability, the virtual mass, moment of inertia, and friction parameters of the endoscope may be controlled to have smaller values. By the control, the burden on the surgeon when directly holding and operating the endoscope device by hand can be reduced. In contrast, at a position with low reliability, the above-described various parameters may be controlled to have larger values. By the control, suppression of an unexpected start of movement can be controlled.

Furthermore, the information regarding the reliability can also be used for speed control regarding movement of the point of action (for example, the endoscope or the like). As a specific example, under a situation where an insertion operation of the endoscope is performed, control may be performed such that the speed regarding the insertion becomes lower in a region (section) with low reliability, and the speed regarding the insertion becomes higher in a region (section) with high reliability. By such control, for example, even under a situation where an organ is moved to a position where a space is present in the constructed environment map, a contact between the endoscope with the organ can be avoided by stopping the insertion operation of the endoscope. In contrast, in a case where the reliability is high, the endoscope can be more quickly moved to a target position.

As the seventh example, the example of evaluating reliability of information of a surrounding space acquired by an imaging unit or the like and controlling generation or update of an environment map according to an evaluation result has been described with reference to FIGS. 24 and 25.

### Eighth Example: Example of Control Using Prediction Model

Next, as an eighth example, an example of a case of evaluating reliability of acquired information regarding a surrounding space using a prediction model constructed on the basis of machine learning will be described. In the present example, an example of a case of constructing a prediction model on the basis of supervised learning and using the constructed prediction model for determination of reliability will be mainly described.

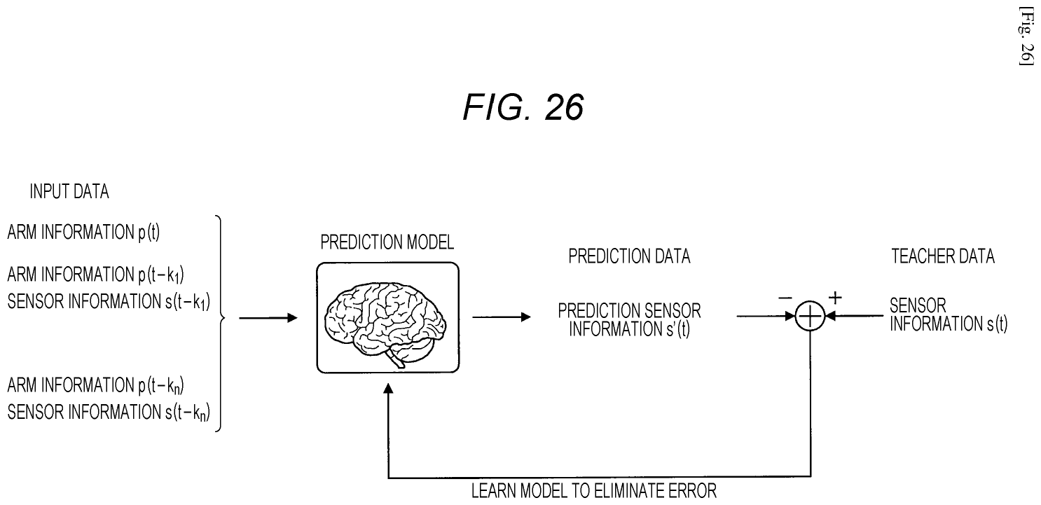

First, an example of a method of constructing a prediction model (AI) will be described with reference to FIG. 26. FIG. 26 is an explanatory diagram for describing an example of control using a prediction model in the medical arm system according to the eighth example, illustrating an example of a method of constructing the prediction model. FIG. 26 illustrates arm information p(t) according to the state of the arm unit 120 at timing t. In other words, p(t−k₁), . . . , p(t−kₙ) represent arm information acquired in the past. Furthermore, information (hereinafter also referred to as “sensor information” for convenience) s(t) is information regarding a surrounding space such as a captured image acquired at the timing t. In other words, s(t−k₁), . . . , s(t−kₙ) represent sensor information acquired in the past.

As illustrated in FIG. 26, in the present example, the arm information and the sensor information acquired in the past are associated with each timing (for example, t−k₁, . . . , t−kₙ) and used as teacher data, and the prediction model (AI) is constructed on the basis of the supervised learning. For example, in a case of machine learning with a multilayer neural network, weighting factors (parameters) among layers of an input layer, an output layer, and a hidden layer of the neural network are adjusted by learning the arm information and the sensor information acquired in the past as learning data, and the prediction model (learned model) is constructed. Then, by inputting the arm information p(t) acquired at the timing t as input data into the prediction model, the prediction model is made to predict sensor information at the timing t. Note that prediction data output as a prediction result at this time is assumed to be prediction sensor information s′(t). Then, an error is calculated on the basis of comparison between the prediction sensor information s′(t) (in other words, the predicted data) output from the prediction model and the sensor information (t) (in other words, the teacher data) actually acquired by the acquisition unit at the timing t, and the error is fed back to the prediction model. In other words, learning is performed to eliminate the error between the prediction sensor information s′(t) and the sensor information (t), so that the prediction model is updated.

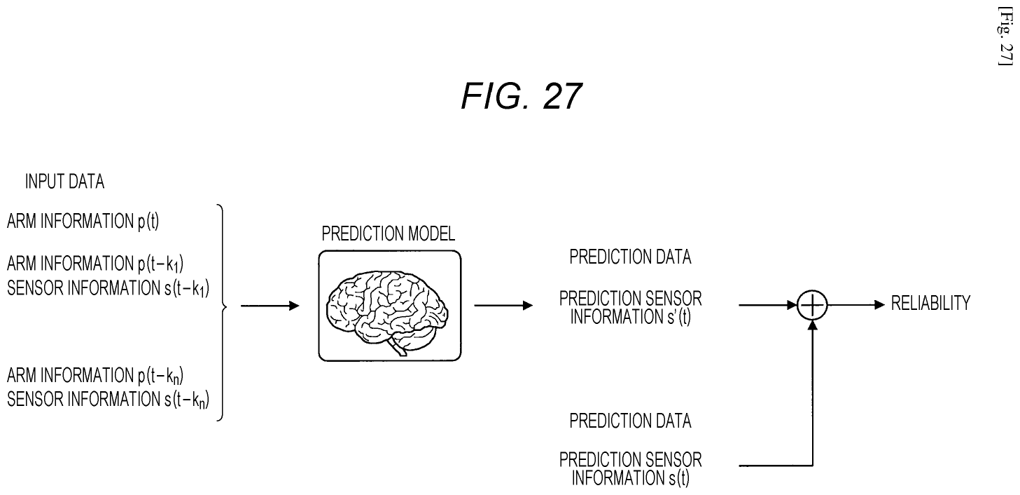

Next, an example of processing regarding determination of reliability of the sensor information using the constructed prediction model will be described with reference to FIG. 27. FIG. 27 is an explanatory diagram for describing an example of control using the prediction model in the medical arm system according to the eighth example, illustrating an example of a method of determining reliability of the sensor information using the prediction model.

As illustrated in FIG. 27, in the present example, the arm information p(t) acquired at the timing t is input to the prediction model constructed on the basis of the arm information and the sensor information acquired in the past, so that the prediction sensor information s′(t) at the timing t is output as the prediction data. Then, the reliability is calculated according to the error between the sensor information s(t) acquired as actual data at the timing t and the prediction sensor information s′(t). In other words, on the premise that the prediction of the prediction model is correct, determination can be made such that the reliability of the sensor information s(t) is lower as the error is larger, and the reliability is higher as the error is smaller.

By use of the determination result of the reliability obtained as described above, information regarding a region where the position and posture of an object are difficult to recognize due to flared highlights or blocked up shadows, for example, can be excluded from the target for the generation or update of the environment map. As a specific example, in a case where flared highlights have occurred due to light reflected by a medical instrument, the region where the reflection has occurred (in other words, the region where flared highlights have occurred) can be excluded from the target for the generation or update of the environment map. Furthermore, in this case, the generation or update of the environment map may be partially performed using information of another portion with high reliability.

Furthermore, as another example, in a case where a state where the reliability is equal to or smaller than a threshold value (in other words, a state where the error between the prediction data and the actual data is equal to or larger than a threshold value) continues beyond a predicted period, the update of the environment map may be performed. By applying such control, occurrence of a situation where generation or update of the environment map is frequently performed due to noise can be prevented.

Note that the information used as the sensor information is not particularly limited as long as the information can be used for the generation or update of the environment map. In other words, as described above, the imaging result by the imaging unit, the measurement result by the distance measurement sensor, the imaging result of the pattern light, the imaging result of the special light, the imaging result by the polarization image sensor, and the like can be used as the sensor information. Furthermore, a plurality of types of information may be used as the sensor information. In this case, for example, the reliability determination may be performed for each type of the sensor information, and the final reliability may be calculated in consideration of the determination result of each reliability.

Furthermore, the accuracy of the prediction by the prediction model can be improved using other information as the learning data. For example, the accuracy of the prediction can be improved by comparing data acquired before surgery by CT, MRI, or the like with data acquired during surgery (for example, the arm information, the sensor information, the prediction sensor information, or the like). Furthermore, information of an environment where the procedure is performed can also be used. As a specific example, change in the posture of the patient's body can be recognized using tilt information of a surgical bed, whereby, for example, change in the shape of the organ according to the change in the posture can be predicted. By use of these pieces of information, deviation of the prediction result by the prediction model according to the situation at that time can be corrected.

As the eighth example, the example of a case of evaluating reliability of acquired information regarding a surrounding space using a prediction model constructed on the basis of machine learning has been described with reference to FIGS. 26 and 27.

### Ninth Example: Presentation of Environmental Map

Next, as a ninth example, presentation of an environment map will be described. A result of generation or update of the environment map may be presented to the operator via an output unit such as a display, for example. At this time, for example, by super-imposing the generated or updated environment map on a human body model, a region where the environment map has been constructed can be presented to the operator. Furthermore, the generated or updated environment map may be superimposed and displayed not only on the human body model but also on so-called preoperative plan information such as a CT image or an MRI image acquired before surgery.

<<6. Hardware Configuration>>

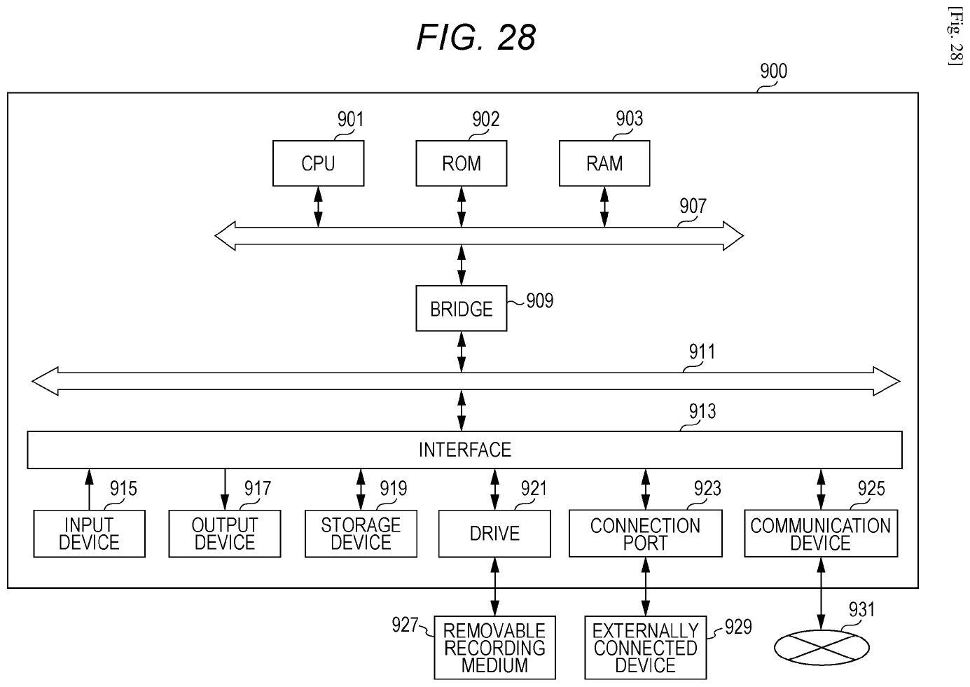

Next, an example of a hardware configuration of an information processing apparatus 900 illustrated in FIG. 28, which configures the medical arm system according to the present embodiment, like the support arm device 10 and the control device 20 according to an embodiment of the present disclosure, will be described. FIG. 28 is a functional block diagram illustrating a configuration example of a hardware configuration of an information processing apparatus according to an embodiment of the present disclosure.

The information processing apparatus 900 according to the present embodiment mainly includes a CPU 901, a ROM 902, and a RAM 903. Furthermore, the information processing apparatus 900 includes a host bus 907, a bridge 909, an external bus 911, an interface 913, a storage device 919, a drive 921, a connection port 923, and a communication device 925. Furthermore, the information processing apparatus 900 may also include at least one of an input device 915 or an output device 917.

The CPU 901 functions as an arithmetic processing unit and a control unit, and controls the entire operation or a part of the information processing apparatus 900 according to various programs recorded in the ROM 902, the RAM 903, the storage device 919, or a removable recording medium 927. The ROM 902 stores programs, operation parameters, and the like used by the CPU 901. The RAM 903 primarily stores the programs used by the CPU 901, parameters that appropriately change in execution of the programs, and the like. The CPU 901, the ROM 903, and the RAM 905 are mutually connected by the host bus 907 configured by an internal bus such as a CPU bus. Note that the arm control unit 110 of the support arm device 10 and the control unit 230 of the control device 20 in the example illustrated in FIG. 6 can be realized by the CPU 901.

The host bus 907 is connected to the external bus 911 such as a peripheral component interconnect/interface (PCI) bus via the bridge 909. Furthermore, the input device 915, the output device 917, the storage device 919, the drive 921, the connection port 923, and the communication device 925 are connected to the external bus 911 via the interface 913.

The input device 915 is an operation unit operated by the user, such as a mouse, a keyboard, a touch panel, a button, a switch, a lever, and a pedal, for example. Furthermore, the input device 915 may be, for example, a remote control unit (so-called remote controller) using infrared rays or other radio waves or an externally connected device 929 such as a mobile phone or a PDA corresponding to an operation of the information processing apparatus 900. Moreover, the input device 915 is configured by, for example, an input control circuit for generating an input signal on the basis of information input by the user using the above-described operation unit and outputting the input signal to the CPU 901, or the like. The user of the information processing apparatus 900 can input various data and give an instruction on processing operations to the information processing apparatus 900 by operating the input device 915.

The output device 917 is configured by a device that can visually or audibly notify the user of acquired information. Such devices include display devices such as a CRT display device, a liquid crystal display device, a plasma display device, an EL display device, a lamp, and the like, sound output devices such as a speaker and a headphone, and a printer device. The output device 917 outputs, for example, results obtained by various types of processing performed by the information processing apparatus 900. Specifically, the display device displays the results of the various types of processing performed by the information processing apparatus 900 as texts or images. Meanwhile, the sound output device converts an audio signal including reproduced sound data, voice data, or the like into an analog signal and outputs the analog signal.

The storage device 919 is a device for data storage configured as an example of a storage unit of the information processing apparatus 900. The storage device 919 is configured by a magnetic storage device such as a hard disk drive (HDD), a semiconductor storage device, an optical storage device, a magneto-optical storage device, or the like. The storage device 919 stores programs executed by the CPU 901, various data, and the like. Note that the storage unit 220 in the example illustrated in FIG. 6 can be realized by, for example, at least one of or a combination of two or more of the ROM 902, the RAM 903, and the storage device 919.

The drive 921 is a reader/writer for a recording medium, and is built in or is externally attached to the information processing apparatus 900. The drive 921 reads out information recorded on the removable recording medium 927 such as a mounted magnetic disk, optical disk, magneto-optical disk, or semiconductor memory, and outputs the information to the RAM 903. Furthermore, the drive 921 can also write a record on the removable recording medium 927 such as the mounted magnetic disk, optical disk, magneto-optical disk, or semiconductor memory. The removable recording medium 927 is, for example, a DVD medium, an HD-DVD medium, a Bluray (registered trademark) medium, or the like. Furthermore, the removable recording medium 927 may be a compact flash (CF (registered trademark)), a flash memory, a secure digital (SD) memory card, or the like. Furthermore, the removable recording medium 927 may be, for example, an integrated circuit (IC) card on which a non-contact IC chip is mounted, an electronic device, or the like.

The connection port 923 is a port for being directly connected to the information processing apparatus 900. Examples of the connection port 923 include a universal serial bus (USB) port, an IEEE 1394 port, a small computer system interface (SCSI) port, and the like. Other examples of the connection port 923 include an RS-232C port, an optical audio terminal, a high-definition multimedia interface (HDMI) (registered trademark) port, and the like. By connecting the externally connected device 929 to the connection port 923, the information processing apparatus 900 directly acquires various data from the externally connected device 929 and provides various data to the externally connected device 929.

The communication device 925 is, for example, a communication interface configured by a communication device for being connected to a communication network (network) 931, and the like The communication device 925 is, for example, a communication card for a wired or wireless local area network (LAN), Bluetooth (registered trademark), a wireless USB (WUSB), or the like. Furthermore, the communication device 925 may be a router for optical communication, a router for an asymmetric digital subscriber line (ADSL), a modem for various communications, or the like. The communication device 925 can transmit and receive signals and the like to and from the Internet and other communication devices in accordance with a predetermined protocol such as TCP/IP, for example. Furthermore, the communication network 931 connected to the communication device 925 is configured by a network or the like connected by wire or wirelessly, and may be, for example, the Internet, home LAN, infrared communication, radio wave communication, satellite communication, or the like.

In the above, an example of the hardware configuration that can realize the functions of the information processing apparatus 900 according to the present embodiment of the present disclosure has been described. Each of the above-described constituent elements may be configured using general-purpose members or may be configured by hardware specialized for the function of each constituent element. Therefore, the hardware configuration to be used can be changed as appropriate according to the technical level of the time of carrying out the present embodiment. Furthermore, although not illustrated in FIG. 28, the information processing apparatus 900 may have various configurations for realizing the function according to the function that can be executed.

Note that a computer program for realizing the functions of the information processing apparatus 900 according to the above-described present embodiment can be prepared and implemented on a personal computer or the like. Furthermore, a computer-readable recording medium in which such a computer program is stored can be provided. The recording medium is, for example, a magnetic disk, an optical disk, a magneto-optical disk, a flash memory, or the like. Furthermore, the above computer program may be delivered via, for example, a network without using a recording medium. Furthermore, the number of computers that execute the computer program is not particularly limited. For example, a plurality of computers (for example, a plurality of servers or the like) may execute the computer program in cooperation with one another.

### 7. Application

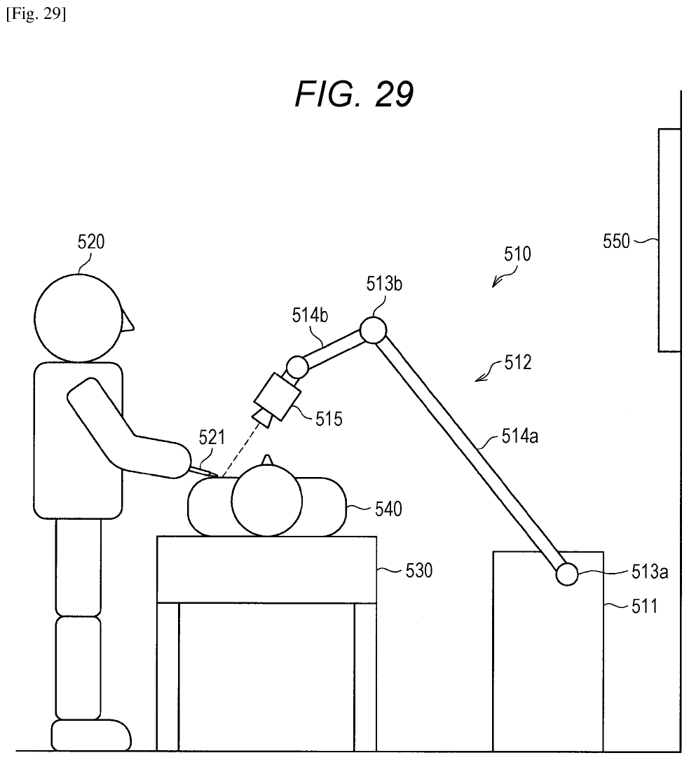

Next, as an application of a medical observation system according to an embodiment of the present disclosure, an example in which the medical observation system is configured as a microscope imaging system including a microscope unit will be described with reference to FIG. 29.

FIG. 29 is an explanatory diagram for describing an application of a medical observation system according to an embodiment of the present disclosure, illustrating an example of a schematic configuration of the microscope imaging system. Specifically, FIG. 29 illustrates, as an application of a case of using the microscope imaging system according to an embodiment of the present disclosure, an example of a case of using a surgical video microscope device provided with an arm will be described.

For example, FIG. 29 schematically illustrates a state of treatment using the surgical video microscope device. Specifically, referring to FIG. 29, a state in which a surgeon who is a practitioner (user) 520 is performing an operation on an operation target (patient) 540 on an operation table 530 using uses a surgical instrument 521 such as a scalpel or forceps is illustrated. Note that, in the following description, the term “operation” is a generic term for various types of medical treatment such as surgery and examination performed by a surgeon as the user 520 for the patient as the operation target 540 Furthermore, the example in FIG. 29 illustrates a state of surgery as an example of the operation, but the operation using a surgical video microscope device 510 is not limited to surgery, and may be used in other various operations.

The surgical video microscope device 510 is provided beside the operation table 530. The surgical video microscope device 510 includes a base unit 511 that is a base, an arm unit 512 extending from the base unit 511, and an imaging unit 515 connected to a distal end of the arm unit 512 as a distal end unit The arm unit 512 includes a plurality of joint units 513a, 513b, and 513c, a plurality of links 514a and 514b connected by the joint units 513a and 513b, and the imaging unit 515 provided at the distal end of the arm unit 512. In the example illustrated in FIG. 29, the arm unit 512 includes the three joint units 513a to 513c and the two links 514a and 514b for the sake of simplicity. However, in reality, the numbers and shapes of the joint units 513a to 513c and the links 514a and 514b, the direction of drive shafts of the joint units 513a to 513c, and the like may be appropriately set in consideration of the degrees of freedom in the positions and postures of the arm unit 512 and the imaging unit 515.

The joint units 513a to 513c have a function to rotatably connect the links 514a and 514b to each other, and the drive of the arm unit 512 is controlled when the rotation of the joint units 513a to 513c is driven. Here, in the following description, the position of each configuration member of the surgical video microscope device 510 means the position (coordinates) in the space defined for drive control, and the posture of each configuration member means the direction (angle) with respect to any axis in the space defined for drive control. Furthermore, in the following description, drive (or drive control) of the arm unit 512 refers to the position and posture of each configuration member of the arm unit 512 being changed (change being controlled) by drive (drive control) of the joint units 513a to 513c and drive (drive control) of the joint units 513a to 513c.

The imaging unit 515 is connected to the distal end of the arm unit 512 as the distal end unit. The imaging unit 515 is a unit that acquires an image of an imaging target object, and is, for example, a camera that can capture a moving image or a still image. As illustrated in FIG. 29, the positions and postures of the arm unit 512 and the imaging unit 515 are controlled by the surgical video microscope device 510 so that the imaging unit 515 provided at the distal end of the arm unit 512 captures a state of the operation site of the operation target 540. Note that the configuration of the imaging unit 515 connected to the distal end of the arm unit 512 as the distal end unit is not particularly limited. For example, the imaging unit 515 is configured as a microscope that acquires an enlarged image of the imaging target object. Furthermore, the imaging unit 515 may be configured to be attachable to and detachable from the arm unit 512. With such a configuration, for example, the imaging unit 515 according to an application may be appropriately connected to the distal end of the arm unit 512 as the distal end unit. Note that, as the imaging unit 515, for example, an imaging device to which the branching optical system according to the above-described embodiment is applied can be applied. In other words, in the present application, the imaging unit 515 or the surgical video microscope device 510 including the imaging unit 515 may correspond to an example of a “medical observation device”. Furthermore, although the description has been made focusing on the case where the imaging unit 515 is applied as the distal end unit, the distal end unit connected to the distal end of the arm unit 512 is not necessarily limited to the imaging unit 515.

Furthermore, at a position facing the user 520, a display device 550 such as a monitor or a display is installed. An image of an operation site captured by the imaging unit 515 is displayed as an electronic image on a display screen of the display device 550. The user 520 performs various types of treatment while viewing the electronic image of the treatment site displayed on the display screen of the display device 550.

With the above-described configuration, the surgery can be performed while imaging the treatment site by the surgical video microscope device 510.

Note that the technology according to the above-described present disclosure can be applied within a range without deviating from the basic idea of the medical observation system according to an embodiment of the present disclosure. As a specific example, the technology according to the above-described present disclosure can be appropriately applied to not only the system to which the above-described endoscope or operation microscope is applied but also a system capable of observing an affected part by capturing an image of the affected part by an imaging device in a desired form.

As the application of the medical observation system according to an embodiment of the present disclosure, the example in which the medical observation system is configured as a microscope imaging system including a microscope unit has been described with reference to FIG. 29.

### 8. Conclusion

As described above, the medical arm system according to an embodiment of the present disclosure includes the arm unit and the control unit. The arm unit is configured to be bendable at least in part, and is configured to be able to support a medical instrument. The control unit controls the operation of the arm unit such that the position and the attitude of the point of action set using at least a part of the arm unit as a reference are controlled. The acquisition unit that acquires the information of a surrounding space is supported by at least a part of the arm unit. The control unit generates or updates the mapping information regarding at least the space around the point of action on the basis of the environment environment information acquired by the acquisition unit and the arm state information regarding the position and posture of the point of action according to the state of the arm unit.

According to the above configuration, the medical arm system according to an embodiment of the present disclosure generates or updates the environment map regarding the external environment of the arm unit (in particular, the environment around the medical instrument or the like supported by the arm unit), and can accurately recognize the position and posture of the observation target using the environment map. In particular, according to the medical arm system according to the present embodiment, the position, posture of the object (for example, the organ or the like) located outside the imaging range of the endoscope device can be recognized using the environment map. Thereby, the medical arm system according to the present embodiment can more accurately control the operation of the arm unit in a more favorable form according to the environment around the arm (for example, the position, the posture of the observation target and the surrounding objects).

Although the favorable embodiments of the present disclosure have been described in detail with reference to the accompanying drawings, the technical scope of the present disclosure is not limited to such examples. It is obvious that persons having ordinary knowledge in the technical field of the present disclosure can conceive various changes and alterations within the scope of the technical idea described in the claims, and it is naturally understood that these changes and alterations belong to the technical scope of the present disclosure.

As a specific example, a device responsible for the generation or update of the environment map and a device responsible for the control of the operation of the arm unit using the environment map may be separately provided. In other words, a certain control device may control the operation of the arm unit associated with the certain control device using the environment map generated or updated by another control device. Note that, in this case, for example, the certain control device and the another control device may mutually recognize the states of the arm units to be respectively controlled by exchanging information regarding the states of the arm units associated with the control devices (for example, the arm information) between the control devices. Thus, the control device on the side using the environment map can recognize the position and posture in the environment map of the medical instrument (in other words, the point of action) supported by the arm unit associated with the control device, according to a relative relationship with the medical instrument supported by the arm unit associated with the control device on the side performing the generation or update of the environment map.

Furthermore, the arm unit supporting the acquisition unit (for example, the endoscope device) that acquires the information regarding the generation or update of the environment map and the arm unit controlled using the environment map may be different. Thus, for example, the environment map is generated or updated on the basis of the information acquired by an endoscope device supported by a certain arm unit, and the operation of another arm unit supporting a medical instrument different from the aforementioned endoscope device may be able to be controlled using the environment map. In this case, the self-position of the medical instrument (endoscope device or the like) supported by each arm can be recognized in accordance with the state (for example, the position and posture) of the arm unit. In other words, by collating the self-position of each medical instrument with the environment map, a relationship of the position and posture between the medical instrument and another object (for example, an organ or the like) located in a space around the medical instrument can be recognized. Of course, even in this case, the operation of the arm unit supporting the acquisition unit can be controlled using the environment map.

Furthermore, in the above description, the arm control according to the present embodiment has mainly been described focusing on the control of the arm unit of the medical arm device. However, the present embodiment does not limit the application destination of the arm control according to the present embodiment (in other words, an application field). As a specific example, the arm control according to an embodiment of the present disclosure can be applied to an industrial arm device. As a more specific example, a working robot provided with the arm unit is brought to enter a region where entry by a person is difficult, and the working robot can be remotely operated. In such a case, the arm control (in other words, the control using the environment map) according to an embodiment of the present disclosure can be applied to the remote control of the arm unit of the working robot.

Furthermore, the effects described in the present specification are merely illustrative or exemplary and are not restrictive. That is, the technology according to the present disclosure can exhibit other effects obvious to those skilled in the art from the description of the present specification together with or in place of the above-described effects.

Note that following configurations also belong to the technical scope of the present disclosure.

(1)

A medical arm system including:

an arm unit configured to support a medical instrument, and to adapt a position and a posture of the medical instrument with respect to a point of action on the medical instrument; and

a control unit configured to control an operation of the arm unit to adapt the position and the posture of the medical instrument with respect to the point of action and one or more acquisition units configured to acquire environment information of a space surrounding the point of action, wherein

the control unit is configured to generate or to update mapping information mapping the space surrounding the point of action on a basis of the environment information acquired by the one or more acquisition units and arm state information representing the position and the posture of the medical instrument with respect to the point of action according to a state of the arm unit.

(2)

The medical arm system according to (1), in which the control unit generates or updates the mapping information on a basis of the environment information and the arm state information, and the arm state information represents a change in at least one of the position or the posture of the medical instrument with respect to the point of action.

(3)

The medical arm system according to (1) or (2), in which the one or more acquisition units include an imaging unit that captures an image of the space surrounding the point of action and generates information representing the image of the space surrounding the point of action, and the control unit generates or updates the mapping information on the basis of the environment information and the arm state information, and the environment information includes the image information of the image captured by the imaging unit.

(4)

The medical arm system according to (3), in which the imaging unit is configured to capture the image of the space surrounding the point of action and generates the image information representing the image of the space surrounding the point of action.

(5)

The medical arm system according to any one of (1) to (4), in which the one or more acquisition units include one or more of an imaging unit, a distance measurement sensor, a polarization image sensor, and an IR image sensor.

(6)

The medical arm system according to (5), in which:

the environment information includes one or more of images generated by the imaging unit, distances measured by the distance measurement sensor, polarized images generated by the polarization image sensor and infrared images generated by the IR image sensor.

(7)

The medical arm system according to (6), including:

a branching optical system configured to partition a light beam incident onto the branching optical system into a plurality of light beams, in which each of the one or more acquisition units individually detects one of the plurality of light beams and uses the detected light beam to acquire the environment information.

(8)

The medical arm system according to (7), in which one or more of the acquisition units is configured to be attachable to and detachable from a housing in which the branching optical system is supported.

(9)

The medical arm system according to any one of (5) to (8), in which at specified time intervals, the imaging unit captures an image of the space surrounding the point of action, each of the images captured by the imaging unit forming part of the environment information.

(10)

The medical arm system according to any one of (1) to (9), in which the medical instrument includes one or more of the one or more acquisition units.

(11)

The medical arm system according to (10), in which the medical instrument includes an endoscope unit including a barrel to be inserted into a body cavity of a patient.

(12)

The medical arm system according to any one of (1) to (11), in which the environment information includes information regarding a space in a body cavity of a patient, and the mapping information is generated or updated on the basis of the environment information and the arm state information.

(13)

The medical arm system according to (12), wherein the information regarding the space in the body cavity of the patient comprises information regarding a site in the body cavity of the patient and information regarding an object in the body cavity, and the control unit excludes the information regarding the object in the body cavity when generating or updating the mapping information.

(14)

The medical arm system according to any one of (1) to (13), in which the control unit determines whether or not to generate or update the mapping information on a basis of the environment information according to a reliability of the environment information.

(15)

The medical arm system according to (14), wherein

the environment information includes image information of an image of the space surrounding a point of action, and

the reliability of the image information is determined according to a brightness of at least a part of the image.

(16)

The medical arm system according to (14), in which the reliability of the image information is determined based on a comparison of the image information with a predicted image information, wherein the predicted image information is generated using a combination of a previous image information of an image of the space surrounding the point of action at an earlier point in time and a previous arm state information representing the position and the posture of the point of action at an earlier point in time.

(17)

The medical arm system according to (16), in which the previous image information and the previous arm state information are training data used to train a machine learning prediction model used to generate the predicted image information.

(18)

The medical arm system according to any one of (1) to (17), in which the arm unit is configured to have a plurality of links rotatable to each other by a joint unit, and the acquisition unit is supported by at least a part of the plurality of links.

(19)

The medical arm system according to (1), in which the control unit controls the operation of the arm unit based on a relative positional relationship between an object specified by the mapping information and the point of action.

(20)

The medical arm system according to (19), in which the control unit controls the operation of the arm unit to generate a reaction force to oppose an external force applied to the arm unit based on a distance between the object specified by the mapping information and the point of action.

(21)

The medical arm system according to (19), in which the control unit controls a moving speed of the arm unit according to a distance between the object and the point of action.

(22)

The medical arm system according to (19), in which the control unit adjusts a maximum movement threshold according to a distance between the object and the point of action, in which the maximum movement threshold defines the maximum allowed adjustment of a position and posture of the arm unit.

(23)

The medical arm system according to (19), in which the control unit controls the operation of the arm unit such that the point of action moves along a surface of the object.

(24)

The medical arm system according to (23), in which the control unit controls the operation of the arm unit such that a change in a posture of the point of action with respect to a normal vector on the surface of the object is limited to fall within a predetermined range.

(25)

The medical arm system according to any one of (19) to (24), in which the control unit controls the operation of the arm unit according to a relative positional relationship between a region where the mapping information has not been generated and the point of action.

(26)

The medical arm system according to (25), in which the control unit controls the operation of the arm unit such that entry of the point of action into the region where the mapping information has not been generated is suppressed.

(27)

The medical arm system according to any one of (1) to (26), in which the control unit is configured to generate or update the mapping information by reconstructing a three dimensional space based on the image information of the image captured by the imaging unit.

(28)

The medical arm system according to any one of (1) to (27), in which the reconstruction of the three dimensional space comprises extracting a plurality of characteristic points from the image of the space surrounding the point of action captured by the imaging unit.

(29)

The medical arm system according to any one of (1) to (28), in which the plurality of characteristic points are one or both of vertexes or edges of objects within the image of the space surrounding the point of action captured by the imaging unit.

(30)

The medical arm system according to any one of (1) to (29), in which the imaging unit captures a plurality of images of the space surrounding the point of action and the reconstruction of the three dimensional space includes extracting a plurality of characteristic points from each of the plurality of images, and reconstructing the three dimensional space on a basis of a correspondence between the plurality of characteristic points of at least one of the plurality of images and the plurality of characteristic points of at least one other of the plurality of images.

(31)

The medical arm system according to any one of (1) to (30), in which the reconstruction of the three dimensional space includes combining the image information of the image of the space surrounding the point of action captured by the imaging unit and the arm state information.

(32)

The medical arm system of any one of (1) to (30), in which the combining of the image information and the arm state information includes calculating mapping parameters to enable mapping between the position and the posture of at least one characteristic point of the plurality of characteristic points in a frame of reference of the captured image and the position and the posture of a corresponding characteristic point in a frame of reference of the arm unit.

(33)

The medical arm system according to any one of (1) to (27), in which the reconstruction of the three dimensional space includes extracting color information from the image of the surrounding space captured by the imaging unit.

(34)

The medical arm system according to any one of (1) to (5), in which the control unit is configured to generate or update the mapping information by reconstructing a three dimensional space using a distance between an object and the distance measurement sensor.

(35)

The medical arm system according to any one of (1) to (5), in which the control unit is configured to generate or update the mapping information by reconstructing a three dimensional space based on a polarized image information of a polarized image captured by the polarization sensor.

(36)

The medical arm system according to any one of (1) to (35), in which the control unit is configured to control the position and posture of the medical instrument with respect to the point of action in response to a user input.

(37)

A control device including:

a control unit configured to control an operation of an arm unit to adapt a position and a posture of a medical instrument with respect to a point of action on the medical instrument, the arm unit being configured to support the medical instrument, and

one or more acquisition units configured to acquire information of a space surrounding the point of action, wherein

the control unit is configured to generate or update mapping information mapping the space surrounding the point of action on a basis of environment information acquired by the one or more acquisition units and arm state information representing the position and the posture of the medical instrument with respect to the point of action according to a state of the arm unit.

(38)

A control device according to (37), wherein

the control unit controls the operation of the arm unit on a basis of mapping information mapping a space surrounding the point of action.

(39)

A control method including:

by a computer,

controlling an arm unit to adapt a position and a posture of a medical instrument with respect to a point of action on the medical instrument, the arm unit being configured to support the medical instrument,

acquiring environment information of a space surrounding the point of action, and

generating or updating mapping information mapping the space surrounding the point of action on a basis of the environment information acquired by the acquisition unit and arm state information representing the position and the posture of the medical instrument with respect to the point of action according to a state of the arm unit.

(40)

A control method according to (39) wherein

the operation of the arm unit is controlled on a basis of mapping information mapping a space surrounding the point of action.

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

1 Medical arm system
10 Support arm device
20 Control device
30 Display device
110 Arm control unit
111 Drive control unit
120 Arm unit
130 Joint unit
131 Joint drive unit
132 Joint state detection unit
133 Rotation angle detection unit
134 Torque detection unit
140 Imaging unit
200 Passive joint unit
210 Input unit
220 Storage unit
230 Control unit
240 Whole body coordination control unit
241 Arm state unit
242 Arithmetic condition setting unit
243 Virtual force calculation unit
244 Real force calculation unit
250 Ideal joint control unit
251 Disturbance estimation unit
252 Command value calculation unit
1000 Endoscope device
1001 Endoscope unit
1003 Camera head
1005 Branching optical system
1007 Imaging unit
1009 Acquisition unit

1. A medical arm system comprising:
an arm unit configured to support a medical instrument, and to adapt a position and a posture of the medical instrument with respect to a point of action on the medical instrument; and
a control unit configured to control an operation of the arm unit to adapt the position and the posture of the medical instrument with respect to the point of action and,
one or more acquisition units configured to acquire environment information of a space surrounding the point of action, wherein
the control unit is configured to generate or to update mapping information mapping the space surrounding the point of action on a basis of the environment information acquired by the one or more acquisition units and arm state information representing the position and the posture of the medical instrument with respect to the point of action according to a state of the arm unit.

2. The medical arm system according to claim 1, wherein the control unit generates or updates the mapping information on a basis of the environment information and the arm state information, and the arm state information comprises a change in at least one of the position or the posture of the medical instrument with respect to the point of action.

3. The medical arm system according to claim 1, wherein
the one or more acquisition units include an imaging unit that captures an image and generates image information representing the image, and
the control unit generates or updates the mapping information on the basis of the environment information and the arm state information, wherein the environment information includes the image information of the image captured by the imaging unit.

4. The medical arm system of claim 3, wherein the imaging unit is configured to capture the image of the space surrounding the point of action and generates the image information representing the image of the space surrounding the point of action.

5. The medical arm system according to claim 1, wherein the one or more acquisition units include one or more of an imaging unit, a distance measurement sensor, a polarization image sensor, and an IR image sensor.

6. The medical arm system according to claim 5, wherein:
the environment information comprises one or more of images generated by the imaging unit, distances measured by the distance measurement sensor, polarized images generated by the polarization image sensor and infrared images generated by the IR image sensor.

7. The medical arm system according to claim 6, comprising:
a branching optical system configured to partition a light beam incident onto the branching optical system into a plurality of light beams, wherein each of the one or more acquisition units individually detects one of the plurality of light beams and uses the detected light beam to acquire the environment information.

8. The medical arm system according to claim 7, wherein one or more of the acquisition units is configured to be attachable to and detachable from a housing in which the branching optical system is supported.

9. The medical arm system according to claim 4, wherein at specified time intervals, the imaging unit captures an image of the space surrounding the point of action, each of the images captured by the imaging unit forming part of the environment information.

10. The medical arm system according to claim 1, wherein the medical instrument includes one or more of the one or more acquisition units.

11. The medical arm system according to claim 10, wherein the medical instrument includes an endoscope unit including a barrel to be inserted into a body cavity of a patient.

12. The medical arm system according to claim 1, wherein the environment information comprises information regarding a space in a body cavity of a patient, and the mapping information is generated or updated on the basis of the environment information and the arm state information.

13. The medical arm system according to claim 12, wherein the information regarding the space in the body cavity of the patient comprises information regarding a site in the body cavity of the patient and information regarding an object in the body cavity, and the control unit excludes the information regarding the object in the body cavity when generating or updating the mapping information.

14. The medical arm system according to claim 1, wherein the control unit determines whether or not to generate or update the mapping information on a basis of the environment information according to a reliability of the environment information.

15. The medical arm system according to claim 14, wherein
the environment information comprises image information of an image of the space surrounding a point of action, and
the reliability of the image information is determined according to a brightness of at least a part of the image.

16. The medical arm system according to claim 14, wherein the reliability of the image information is determined based on a comparison of the image information with a predicted image information, wherein the predicted image information is generated using a combination of a previous image information of an image of the space surrounding the point of action at an earlier point in time and a previous arm state information representing the position and the posture of the point of action at an earlier point in time.

17. The medical arm system according to claim 16, wherein the previous image information and the previous arm state information are training data used to train a machine learning prediction model used to generate the predicted image information.

18. The medical arm system according to claim 1, wherein
the arm unit is configured to have a plurality of links rotatable to each other by a joint unit, and
the acquisition unit is supported by at least a part of the plurality of links.

19. The medical arm system according to claim 1, wherein the control unit controls the operation of the arm unit based on a relative positional relationship between an object specified by the mapping information and the point of action.

20. The medical arm system according to claim 19, wherein the control unit controls the operation of the arm unit to generate a reaction force to oppose an external force applied to the arm unit based on a distance between the object specified by the mapping information and the point of action.

21.-40. (canceled)
